(19) United States
(12) Patent Application Publication
(10) Pub. No.: US 2024/0013853 A1
(43) Pub. Date: Jan. 11, 2024

(54) De Novo Designed Homo-Oligomeric Protein Assemblies

(71) Applicant: University of Washington, Seattle, WA (US)

(72) Inventors: David BAKER, Seattle, WA (US); Basile WICKY, Seattle, WA (US); Lukas MILLES, Seattle, WA (US); Alexis COURBET, Seattle, WA (US); Robert RAGOTTE, Seattle, WA (US); Elias KINFU, Seattle, WA (US); Sam TIPPS, Seattle, WA (US); Justas DAUPARAS, Seattle, WA (US); Ryan KIBLER, Seattle, WA (US)

(21) Appl. No.: 18/348,528

(22) Filed: Jul. 7, 2023

Related U.S. Application Data
(60) Provisional application No. 63/368,093, filed on Jul. 11, 2022.

(51) Int. Cl. G16B 15/20

(52) U.S. Cl. CPC G16B 15/20

(57) ABSTRACT

Polypeptide are providing that are at least 50% identical to the amino acid sequence selected from the group consisting of SEQ ID NOS:1-37, cyclic homo-oligomers of the polypeptides, and uses thereof.

### CROSS REFERENCE

This application claims priority to U.S. Provisional Patent Application Ser. No. 63/368,093 filed Jul. 11, 2022, incorporated by reference herein in its entirety.

### FEDERAL FUNDING STATEMENT

This invention was made with government support under Grant No. P41 GM 103533-24, awarded by the National Institute of General Medical Sciences and Grant No. CHE-1629214, awarded by the National Science Foundation. The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Jul. 2, 2023, is named “22-0950-US_Sequence-Listing.xml” and is 108,438 bytes in size.

### BACKGROUND

Cyclic protein oligomers play key roles in almost all biological processes and have many applications, ranging from small molecule binding and catalysis to building blocks for nanocage assemblies, Current approaches to designing cyclic protein oligomers require specification of the structure of the protomers in advance, and with the exception of parametrically designed helical bundles, have involved rigid body docking of previously characterized monomers into higher order symmetric structures followed by interface optimization to confer low energy to the assembled state. The requirement that the protomer structure be specified in advance has limited exploration of the full space of oligomeric structures; in particular assemblies in which the chains are more intertwined.

### SUMMARY

In one aspect, the disclosure provides polypeptides comprising an amino acid sequence at least 50% identical to the amino acid sequence selected from the group consisting of SEQ ID NOS:1-38, wherein any N-terminal amino acid is optional and may be present or may be deleted. In another embodiment, at least 50% of substitutions relative to the reference amino acid sequence are at surface residues as defined in Table 1. In another embodiment, at least 50% of core residues, as defined in Table 1 are maintained as in the reference amino acid sequence. In another embodiment, the polypeptide further comprises one or more functional domains, such as in a fusion protein. In a further embodiment, the disclosure provides cyclic homo-oligomers, comprising one or a plurality of the polypeptides of the disclosure. In one embodiment, the cyclic homo-oligomer comprises a plurality of identical polypeptides of the disclosure. In another embodiment, the cyclic homo-oligomer comprises an amino acid sequence at least 50% identical to the amino acid sequence selected from SEQ ID NO:1-5 and 39-71.

The disclosure also provides nucleic acids encoding the polypeptide or fusion protein of any embodiment herein, expression vectors comprising the nucleic acids of the disclosure operatively linked to a suitable control sequence, and host cells comprising a polypeptide, fusion protein, cyclic homo-oligomer, nucleic acid, or expression vector of the disclosure.

The disclosure also provides methods for use of the polypeptides, fusion proteins, and cyclic homo-oligomers of the disclosure, including but not limited to methods for generating an immune response.

### DESCRIPTION OF THE FIGURES

FIG. 1. Hallucinating protein assemblies (A) Starting from the definition of a cyclic symmetry and protein length, a random sequence is optimized by MCMC through the AF2 network until the resulting structure fits the design objective, followed by sequence re-design with ProteinMPNN. (B) The method generates structurally diverse outputs, quantified here by multi-dimensional scaling of protomer pairwise structural similarities between experimentally tested HALs (N=351) and all de novo cyclic oligomers present in the PDB (N=162). (C) Generated structures are significantly different from anything present in the PDB. Median™-scores to the closest match: 0.67 and 0.57 for the protomers and oligomers respectively (vertical lines). (D) Generated sequences are unrelated to naturally-occuring proteins. Median BLAST E-values from the closet hit in UniRef100: 2.6 and 1.3 for the repeat motifs and protomers respectively (vertical lines). (E) Success counts of ProteinMPNN-designed HALs at different levels of characterization. (F) Most soluble HALs have SEC retention volumes consistent with their oligomeric state. The line shows the fit to calibration standards (open circles), and the shaded area represents the 95% confidence interval of the calibration. (G) Parity plot between the theoretical and observed molecular weights of HALs from SEC-MALS. (H) ProteinMPNN-designed HALs are thermostable. Parity plot between pre-melting and post-melting retention volumes; circles represent designs that remained monodisperse, while triangles indicate polydispersity after heating the sample. In plots E-H, the data is categorized by symmetries. The legend is shown in H.

FIG. 2. Structures of HALs solved by X-ray crystallography compared to their design models. (A) HALC2_062 (RMSD: 0.81 Å). (B) HALC2_065 (RMSD: 1.02 Å). (C) HALC2_068 (RMSD: 0.86 Å). (D) HALC3_104 (RMSD: 0.42 A). (E) HALC3_109 (RMSD: 0.46 Å). (F) HALC4_135 (RMSD: 0.60 Å). (G) HALC4_136 (RMSD: 0.34 Å). In each row, the first panel shows a surface rendering of the oligomer with one protomer highlighted, the second panel, the 2mFo-DFc map compared to the side-chain rotamers of the design model, and the last two panels, two different orientations of the structural overlays between the model and the solved structure.

FIG. 3. Cryo-electron and negative stain electron microscopy validation of large HALs. For each design, the model is shown by chain and the corresponding internal symmetry (X) and oligomerization state (Y) are indicated (CX-Y). The electron density map is shown next to the model alongside characteristic 2D class averages. (A) Negative stain characterization of HALs. Ring diameters are 92 Å, 110 Å, 75 Å, 80 Å, 100 Å, 107 Å, for HALC6_220, HALC24-6_316, HALC20-5_308, HALC25-5_341, HALC18-6_278 and HALC42-7_351, respectively. (B) CryoEM characterisation of three large HALs. The ring diameters are 87 Å, 99 Å, and 100 Å for HALC15-5_262, HALC18-6_265, and HALC33-3_343, respectively. Top row left panels: design model by chain; Top row, right panels: superpositions of the CryoEM model and design model. Bottom row: 4.38 Å, 6.51 Å, and 6.32 Å cryoEM electron density maps. Scale bars=10 nm.

FIG. 4. Hallucinated structures differ significantly from their closest matches in the PDB. For each structure solved by crystallography (FIG. 2) or cryoEM (FIG. 3B), the closest structural match to the protomer and to the oligomer are shown on the left and right respectively. Designs are shown by chain and the closest matching PDB is shown. In most cases the closest oligomer has an entirely different structure; this is particularly evident for the larger designs in G-H. TM-scores (protomerloligomer) are indicated in parentheses, and the PDB IDs are reported in Table 2. (A) HALC2_062 (0.6910.59). (B) HALC2_065 (0.6710.54). (C) HALC2_068 (0.6710.57). (D) HALC3_104 (0.8710.88). (E) HALC3_109 (0.7810.69). (F) HALC4_135 (0.8010.59). (G) HALC4_136 (0.8010.71). (H) HALC15-5_262 (0.6510.46). (I) HALC18-6_265 (0.6510.49). (J) HALC33-3_343 (0.4910.41).

FIG. 5. Soluble yield of AF2 and MPNN designed sequences for small HALs. Bottom plot shows the total soluble protein yield per liter equivalent calculated from integrating the SEC traces (and normalizing by the sequence-specific extinction coefficients) for the original AF2 designs, compared to their MPNN redesigns. In some cases more than one MPNN sequence per backbone was ordered. The top plot summarizes the difference in yield: for the AF2 designs a median yield of 9 mg per L eq. as compared to 247 mg per L eq. for the MPNN sequences.

FIG. 6. SEC elution profiles of small HALs. Samples were run on a Superdex™ 200 increase 10/300 GL following IMAC purification. The results are shown ordered by oligomeric symmetry.

FIG. 7. Characterization of HALs. The first column shows the SEC elution profile (Superdex™ 200 increase 10/300 GL) after IMAC (gray con line, and after heating the sample to 95° C. (dotted line). The second column shows the CD spectra at 25° C. (line), at 95° C. (dashed line) and after cooling back to 25° C. (dotted line). The third column shows the circular dichroic signal at 222 nm during temperature ramping.

FIG. 8. Comparison between AF2 models and crystallographic structures. (A) For each design, five models (one for each ptm model, 10 recycles) were compared to the biounit. If multiple biounits were present, alignments against all bionunits are shown. Alignments were generated using MMalign, and the median RMSD for each design is indicated by a horizontal line. Models that were more confidently predicted (higher pTM values) were closer to the experimentally-validated structures as shown by the bar. (B) The pTM value from each AF2 model correlates with the actual TM-score (from MMalign) between design and structures. The parity is indicated by a line. (C) Structural matching between chains of the asymmetric unit of each design. Pairwise alignments and RMSD values were computed with TMalign, and the median is indicated by a horizontal line. Designs lacking data points only contained one chain in the asymmetric unit.

FIG. 9. RoseTTAFold2 accurately predicts structures of crystallized HALs but not necessarily the original AF2 hallucinated backbone sequence. RoseTTAFold2 predictions compared to the original AF2 hallucination (left). RoseTTAFold2 prediction for the MPNN re-designs of the same backbones (right). (A) HALC2_062 (RMSD: 2.75 Å|0.83 Å). (B) HALC2_065 (RMSD: 4.28 Å|11.11 Å). (C) HALC2_068 (RMSD: 3.91 Å|0.92 Å). (D) HALC3_104 (RMSD: 0.27 Å|10.42 Å). (E) HALC3_109 (RMSD: 0.48 Å|0.55 Å). (F) HALC4_135 (RMSD: 4.08 Å|10.72 Å). (G) HALC4_136 (RMSD: 0.91 Å|0.37 Å). The AF2/crystal structures are shown by chain, and the RoseTTAFold2 predictions are also shown.

FIG. 10. Design models and corresponding experimental negative stain electron microscopy analysis of designs shown in FIG. 3A. A raw micrograph at 57k magnification is shown along with nine example extracted particles that were used for further classification and data processing. From top left to bottom right: HALC6_220, HALC24-6_316, HALC20-5_308, HALC25-5_341, HALC18-6_278 and HALC42-7_351.

FIG. 11. Detailed comparison of HAL designs versus cryoEM structures. The designs were relaxed into experimental cryoEM electron densities using Rosetta FastRelax and SetupForDensityScoring. From Top to Bottom: HALC15-5_262, RUC18-6_265, and HALC33-3_343 Superposition of the designed backbone and backbone relaxed into the experimental electron density. The computed backbone atom RMSD between the designed and experimental structure are 0.81 Å, 1.69 Å, and 2.30 Å respectively.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M.P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V). All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In one aspect, the disclosure provides polypeptides comprising or consisting of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-38, wherein any N-terminal amino acid is optional and may be present or may be deleted.

The polypeptides of the disclosure are capable of forming cyclic homo-oligomers, and thus may be used, for example, in small molecule binding and catalysis, as building blocks for nanocage assemblies, scaffolding of protein binders and building nanomaterials, and for scaffolding antigens for generating an immune response against the antigen. Sequences of the polypeptides are provided in Table 1. In the table, “Sym” means “symmetry”, and “p-Sym” means “pseudosymmetry” (number of chains).

TABLE 1

| | | | | surface residues (lowercase = surface, |
|---|---|---|---|---|
| | | | | uppercase = core) |
| | | | | In this column, the sequence includes the |
| | | P- | | number of copies of the chain as noted in |
| Name | Sym | Sym | sequence | the pseudosymmetry (P-Sym) column |
| HALC1_ | C1 | C1 | MIVSLEKHPGGVHII | miVslekhpggvHiItLsseeNLeNFVkELkkLgAeVerLpe |
| 004 | | | TLSSEENLENFVKEL | pNtVrVrApeeVVeeAlkNTkFk (SEQ ID NO: 1) |
| | | | KKLGAEVERLPEPNT | |
| | | | VRVRAPEEVVEEALK | |
| | | | NTKFK (SEQ ID | |
| | | | NO: 1) | |
| HALC1_ | C1 | C1 | NEKEFLLQLKEELDK | nekefLlqLkeELdkdpseeNVlsLiktLneeQkkILeeIkk |
| 005 | | | DPSEENVLSLIKTLN | kYpnLpLSkIFeLLIdELlerLe (SEQ ID NO: 2) |
| | | | EEQKKILEEIKKKYP | |
| | | | NLPLSKIFELLIDEL | |
| | | | LERLE (SEQ ID | |
| | | | NO: 2) | |
| HALC1_ | C1 | C1 | DKIAFFKRLKEELEK | dkiafFkrLkeELekdpsdeNVekLIeTLneeEkkILeeIkk |
| 006 | | | DPSDENVEKLIETLN | eYpnEpLSeIFyKLIeKLlelSe (SEQ ID NO: 3) |
| | | | EEEKKILEEIKKEYP | |
| | | | NEPLSEIFYKLIEKL | |
| | | | LELSE (SEQ ID | |
| | | | NO: 3) | |
| HALC1_ | C1 | C1 | MLSPEELLEKLKKYL | mLspeeLleKLkkYLkekYnVvVpeeRiVsveAtdssVkLtW |
| 007 | | | KEKYNVVVPEERIVS | sRgdgreGtAyYsDegeVrVedP (SEQ ID NO: 4) |
| | | | VEATDSSVKLTWSRG | |
| | | | DGREGTAYYSDEGEV | |
| | | | RVEDP (SEQ ID | |
| | | | NO: 4) | |
| HALC1_ | C1 | C1 | MLTPEELLERLRRHL | mLtpeeLleRLrrHLeeEHgVvVpeeRiLsveAtpteVtLtW |
| 008 | | | EEEHGVVVPEERILS | sRgdgrtGtArYtSdgrFeVeDP (SEQ ID NO: 5) |
| | | | VEATPTEVTLTWSRG | |
| | | | DGRTGTARYTSDGRF | |
| | | | EVEDP (SEQ ID | |
| | | | NO: 5) | |
| HALC2_ | C2 | C2 | MVKPITEEDVREAAT | mvkpIteeDVreAAtaASpdYeVGeAKlIDeenNLWFVtLyk |
| 059 | | | AASPDYEVGEAKLID | gdqkiYALIeDkngeFtVHqIeLmvkpIteeDVreAAtaASp |
| | | | EENNLWFVTLYKGDQ | dYeVGeAKlIDeenNLWFVtLykgdqkiYALIeDkngeFtVH |
| | | | KIYALIEDKNGEFTV | qIeL (SEQ ID NO: 39) |
| | | | HQIEL (SEQ ID | |
| | | | NO: 6) | |
| HALC2_ | C2 | C2 | MARVEYSYEKLNDTH | mArVeYsYekLndthYkLkLkVtYeYrkSpEArrLAeDLVqA |
| 062 | | | YKLKLKVTYEYRKSP | FVdALssLpFItVeYeVeevevemArVeYsYekLndthYkLk |
| | | | EARRLAEDLVQAFVD | LkVtYeYrkSpEArrLAeDLVqAFVdALssLpFItVeYeVee |
| | | | ALSSLPFITVEYEVE | veve (SEQ ID NO: 40) |
| | | | EVEVE (SEQ ID | |
| | | | NO: 7) | |
| HALC2_ | C2 | C2 | MKVYEFPYPETGKKI | MkvYeFpYpETgKkIIVIQGekNIVIVVGnTAVVYYegkWTY |
| 063 | | | IVIQGEKNIVIVVGN | KenVteeDIekAkteeGAkeLAkMkvYeFpYpETgKkIIVIQ |
| | | | TAVVYYEGKWTYKEN | GekNIVIVVGnTAVVYYegkWTYKenVteeDIekAkteeGAk |
| | | | VTEEDIEKAKTEEGA | eLAk (SEQ ID NO: 41) |
| | | | KELAK (SEQ ID | |
| | | | NO: 8) | |
| HALC2_ | C2 | C2 | SKLKEQEELIDEISE | sklkeQeeLIdeISeKAkeFLlEIkekYpGeLseerypgrVv |
| 064 | | | KAKEFLLEIKEKYPG | LtYvNeeKgFsItVtIeLLnkeksklkeQeeLIdeISeKAke |
| | | | ELSEERYPGRVVLTY | FLlEIkekYpGeLseerypgrVvLtYvNeeKgFsItVtIeLL |
| | | | VNEEKGFSITVTIEL | nkek (SEQ ID NO: 42) |
| | | | LNKEK | |
| | | | (SEQ ID NO: 9) | |
| HALC2_ | C2 | C2 | SEEEKPIVIDLNKTI | seeEkpIvIdLnKtIerdgRkVkLvrAtItVdPetNtItIdI |
| 065 | | | ERDGRKVKLVRATIT | eYeGGpItkeDLlEAFkLAAsKLseeEkpIvIdLnKtIerdg |
| | | | VDPETNTITIDIEYE | RkVkLvrAtItVdpetNtItIdIeYeGGpItkeDLlEAFkLA |
| | | | GGPITKEDLLEAFKL | AsKL (SEQ ID NO: 43) |
| | | | AASKL (SEQ ID | |
| | | | NO: 10) | |
| HALC2_ | C2 | C2 | DKLVRVLSSSMIYYA | dkLvrVLSSSMIYYAeRMTkgStdpsDYdkALdDFYnYFleQ |
| 067 | | | ERMTKGSTDPSDYDK | pFVdkeTLekAYeLArkRLeelLdkLvrVLSSSMIYYAeRMT |
| | | | ALDDFYNYFLEQPFV | kgStdpsDYdkALddFYnYFleQpFVdkeTLekAYeLArkRL |
| | | | DKETLEKAYELARKR | eelL (SEQ ID NO: 44) |
| | | | LEELL (SEQ ID | |
| | | | NO: 11) | |
| HALC2_ | C2 | C2 | MIKVPEDLERIGREL | mIkvpeDLerIGreLrargLdTkrLLeeGpkLYpeLSIPDLM |
| 068 | | | RARGLDTKRLLEEGP | AIALYDHLnLdPeFLYrLLqQSrmIkvpeDLerIGreLrarg |
| | | | KLYPELSIPDLMAIA | LdTkrLLeeGpkLYpeLSIPDLMAIALYDHLnLdPeFLYrLL |
| | | | LYDHLNLDPEFLYRL | qQSr (SEQ ID NO: 45) |
| | | | LOQSR (SEQ ID | |
| | | | NO: 12) | |
| HALC3_ | C3 | C3 | LEELKERVEQLEKRL | leeLkeRVeqLekRLSVVESTLTHLLTTFsdeTLkwIYdNTr |
| 100 | | | SVVESTLTHLLTTFS | aDpsVDkeTLdeFWkRVeeEKkkleeLkeRVeqLekRLSVVE |
| | | | DETLKWIYDNTRADP | STLTHLLTTFsdeTLkwIydNTraDpsVDkeTLdeFWkRVee |
| | | | SVDKETLDEFWKRVE | EKkkleeLkeRVeqLekRLSVVESTLTHLLTTFsdeTLkwIY |
| | | | EEKKK | dNTraDpsVDkeTLdeFWkRVeeEKkk (SEQ ID NO: 46) |
| | | | (SEQ ID NO: 13) | |
| HALC3_ | C3 | C3 | KRIDEIESKLKHLEE | krideIesKLkHLeeFTtHLIkLMeTMLeLLkLVSdgkSdse |
| 104 | | | FTTHLIKLMETMLEL | eYkeLLekAeeYLkqAteAAkkIkrideIesKLkHLeeFTtH |
| | | | LKLVSDGKSDSEEYK | LIkLMeTMLeLLkLVSdgkSdseeYkeLLekAeeYLkqAteA |
| | | | ELLEKAEEYLKQATE | AkkIkrideIesKLkHLeeFTtHLIkLMeTMLeLLkLVSdgk |
| | | | AAKKI (SEQ ID | SdseeYkeLLekAeeYLkqAteAAkkI (SEQ ID NO: 47) |
| | | | NO: 14) | |
| HALC3_ | C3 | C3 | MEPEELERLRELYEV | MepeEleRLreLYeVFkdKLdePIGLYLLTLLAIYDperree |
| 105 | | | FKDKLDEPIGLYLLT | YLeKLRdIFekqgetdIAeRLkeMepeEleRLreLYeVFkdK |
| | | | LLAIYDPERREEYLE | LdePIGLYLLTLLAIYDperreeYLeKLRdIFekqgEtdIAe |
| | | | KLRDIFEKQGETDIA | RLkeMelpeEleRLreLYeVFkdKLdePIGLYLLTLLAIYDp |
| | | | ERLKE (SEQ ID | erreeYLeKLRdIFekqgetdIAeRLke (SEQ ID |
| | | | NO: 15) | NO: 48) |
| HALC3_ | C3 | C3 | REEIEEAVKEAELKV | reeIeeAVkeAELKVLAIVLVALRSVshYePLsRLYeSFldA |
| 109 | | | LAIVLVALRSVSHYE | LkKALseeElkEVekEAerIekKreeIeeAVkeAELKVLAIV |
| | | | PLSRLYESFLDALKK | LVALRSVshYePLSRLYeSFldALkKALseeElkEVekEAer |
| | | | ALSEEELKEVEKEAE | IekKreeIeeAVkeAELKVLAIVLVALRSVshYePLsRLYeS |
| | | | RIEKK (SEQ ID | FldALkKALseeELkEVekEAerIekK (SEQ ID NO: 49) |
| | | | NO: 16) | |
| HALC3_ | C3 | C3 | LEQILEELTELLERV | LeqIleELteLLerVdeIpLreALkRMLeLLVRVTqELKeVK |
| 110 | | | DEIPLREALKRMLEL | dKVesLekHLeeLdkRVeeIekkLeqIleELteLLerVdeIp |
| | | | LVRVTQELKEVKDKV | LreALkRMLeLLVRVTqELKeVKdKVesLekHLeeLdkRVee |
| | | | ESLEKHLEELDKRVE | IekkLeqIleELteLLerVdeIpLreALkRMLeLLVRVTqEL |
| | | | EIEKK (SEQ ID | KeVKdKVesLekHLeeLdkRVeeIekk (SEQ ID NO: 50) |
| | | | NO: 17) | |
| HALC3_ | C3 | C3 | MAKLLPGLSEEEKRL | mAkLLpgLseEEkRLTdILDkLLpgLeVlDVLrEdGLVVFLA |
| 111 | | | TDILDKLLPGLEVLD | rHgdHLLVASFTRFkDpeLqsKVmAkLLpgLseEEkRLTdIL |
| | | | VLREDGLVVFLARHG | DkLLpgLeVlDVLrEdGLVVFLArHgdHLLVASFTRFkDpeL |
| | | | DHLLVASFTRFKDPE | qsKVmAkLLpgLseEEkRLTdILDkLLpgLeVlDVLrEdGLV |
| | | | LQSKV (SEQ ID | VFLArHgdHLLVASFTRFkDpeLqsKV (SEQ ID NO: 51) |
| | | | NO: 18) | |
| HALC3_ | C3 | C3 | MTKLVEYHYDEETQL | mTkLVeYhYdeeTQLLYIkLqLnenEyLVLFLYSKeDeeSlk |
| 112 | | | LYIKLQLNENEYLVL | KLkeLeeeAasdpsLHLVKGfFkmTkLVeYhYdeeTQLLYIk |
| | | | FLYSKEDEESLKKLK | LqLnenEyLVLFLYSKeDeeSlkKLkeLeeeAasdpsLHLVK |
| | | | ELEEEAASDPSLHLV | GfFkmTkLVeYhYdeeTQLLYIkLqLnenEyLVLFLYSKeDe |
| | | | KGFFK (SEQ ID | eSlkKLkeLeeeAasdpsLHLVKGfFk (SEQ ID NO: 52) |
| | | | NO: 19) | |
| HALC3_ | C3 | C3 | VDEKEVKERFEEIES | VdekeVkeRFeeIesRLeeLesKVreVekKVeeVkkeSdeKI |
| 114 | | | RLEELESKVREVEKK | dqLkteFetKYnqInnEIntLknVdekeVkeRFeeIesRLee |
| | | | VEEVKKESDEKIDQL | LesKVreVekKVeeVkkeSdeKIdqLkteFetKYnqInnEIn |
| | | | KTEFETKYNQINNEI | tLknVdekeVkeRFeeIesRLeeLesKVreVekKVeeVkkeS |
| | | | NTLKN (SEQ ID | deKIdqLkteFetKYnqInnEIntLkn (SEQ ID NO: 53) |
| | | | NO: 20) | |
| HALC3_ | C3 | C3 | MTRLEQLLAQGVDPF | mTRLeqLlaqgvdPFeVLreKIekLkeIWkkYeeAkgeeker |
| 118 | | | EVLREKIEKLKEIWK | YRdELLkLMMeVLeLMVELLSrRmTRLeqLlaqgvdPFeVLr |
| | | | KYEEAKGEEKERYRD | eKIekLkeIWkkYeeAkgeekerYRdELLkLMMeVLeLMVEL |
| | | | ELLKLMMEVLELMVE | LSrRmTRLeqLlaqgvdPFeVLreKIekLkeIWkkYeeAkge |
| | | | LLSRR (SEQ ID | ekerYRdELLkLMMeVLeLMVELLSrR (SEQ ID NO: 54) |
| | | | NO: 21) | |
| HALC4_ | C4 | C4 | MEKFKEQLLEEVKKI | mekfKeqLLeEVkkIVLeTMTKVMeHLEKWFvTLAeIIItks |
| 135 | | | VLETMTKVMEHLEKW | eeKLeeLkeTMekSIeeLrkEAemekfKeqLLeEVkkIVLeT |
| | | | FVTLAEIIITKSEEK | MTKVMeHLEKWFvTLAeIIItkseeKLeeLkeTMekSIeeLr |
| | | | LEELKETMEKSIEEL | kEAemekfKeqLLeEVkkIVLeTMTKVMeHLEKWFvTLAeII |
| | | | RKEAE (SEQ ID | ItkseeKLeeLkeTMekSIeeLrkEAemekfKeqLLeEVkkI |
| | | | NO: 22) | VLeTMTKVMeHLEKWFvTLAeIIItkseeKLeeLkeTMekSI |
| | | | | eeLrkEAe (SEQ ID NO: 55) |
| HALC4_ | C4 | C4 | MSPYKKAIEITKRLL | mSPYkKAIeITkrLLeLLLsnpeLAkkNLGGIATLISLLALI |
| 136 | | | ELLLSNPELAKKNLG | SALDgtLdekDIepYIkKLeeSLmSPYkKAIeITkrLLeLLL |
| | | | GIATLISLLALISAL | snpeLAkkNLGGIATLISLLALISALDgtLdekDIepYIkKL |
| | | | DGTLDEKDIEPYIKK | eeSLmSPYkKAIeITkrLLeLLLsnpeLAkkNLGGIATLISL |
| | | | LEESL (SEQ ID | LALISALDgtLdekDIepYIkKLeeSLmSPYkKAIeITkrLL |
| | | | NO: 23) | eLLLsnpeLAkkNLGGIATLISLLALISALDgtLdekDIepY |
| | | | | IkKLeeSL (SEQ ID NO: 56) |
| HALC4_ | C4 | C4 | MEEVVLTSHNELHKK | meeVVltSHneLhkKLdeVHdkImsKLdeIheKLdeIisKLd |
| 140 | | | LDEVHDKIMSKLDEI | eIEsKLheILnIVkeIKeILekKmeeVVltSHneLhkKLdeV |
| | | | HEKLDEIISKLDEIE | HdkImsKLdeIheKLdeIisKLdeIEsKLheILnIVkeIKeI |
| | | | SKLHEILNIVKEIKE | LekKmeeVVltSHneLhkKLdeVHdkImsKLdeIheKLdeIi |
| | | | ILEKK (SEQ ID | sKLdeIEsKLheILnIVkeIKeILekKmeeVVltSHneLhkK |
| | | | NO: 24) | LdeVHdkImsKLdeIheKLdeIisKLdeIEsKLheILnIVke |
| | | | | IKeILekK (SEQ ID NO: 57) |
| HALC5_ | C5 | C5 | SSLKEWLERWREKLV | SsLkeWLerWRekLveAVkgTpEeeKVeKYLdLAleSLeEMP |
| 167 | | | EAVKGTPEEEKVEKY | DkkLAeRIASRLFTEAVkTVVeASsLkeWLerWRekLveAVk |
| | | | LDLALESLEEMPDKK | gTpEeeKVeKYLdLAleSLeEMPDkkLAeRIASRLFTEAVkT |
| | | | LAERIASRLFTEAVK | VVeASsLkeWLerWRekLveAVkgTpEeeKVeKYLdLAleSL |
| | | | TVVEA (SEQ ID | eEMPDkkLAeRIASRLFTEAVkTVVeASsLkeWLerWRekLv |
| | | | NO: 25) | eAVkgTpEeeKVeKYLdLAleSLeEMPDkkLAeRIASRLFTE |
| | | | | AVkTVVeASsLkeWLerWRekLveAVkgTpEeeKVeKYLdLA |
| | | | | leSLeEMPDkkLAeRIASRLFTEAVkTVVeA (SEQ ID |
| | | | | NO: 58) |
| HALC5_ | C5 | C5 | LLLEVMEKVFDEEQL | LLleVMekVFdeeQLkLIkeAAerEgnsPvVISSIATLLLLE |
| 169 | | | KLIKEAAEREGNSPV | RIEKIVkeIHdEVkkNNeKQekkLLleVMekVFdeeQLkLIk |
| | | | VISSIATLLLLERIE | eAAerEgnsPvVISSIATLLLLERIEkIVkeIHdEVkkNNeK |
| | | | KIVKEIHDEVKKNNE | QekkLLleVMekVFdeeQLkLIkeAAerEgnsPvVISSIATL |
| | | | KQEKK (SEQ ID | LLLERIEkIVkeIHdEVkkNNeKQekkLLleVMekVFdeeQL |
| | | | NO: 26) | kLIkeAAerEgnsPvVISSIATLLLLERIEkIVkeIHdEVkk |
| | | | | NNeKQekkLLleVMekVFdeeQLkLIkeAAerEgnsPvVISS |
| | | | | IATLLLLERIEkIVkeIHdEVkkNNeKQekk (SEQ ID |
| | | | | NO: 59) |
| HALC5_ | C5 | C5 | RPRPPIRLEVLIEAD | rprPpIrLeVlIeAdLsDpdSLlRAIeEAerTLeRLerDLpp |
| 172 | | | LSDPDSLLRAIEEAE | eVLerFrpHLrLeIlLkKdIkperprPpIrLeVlIeAdLsDp |
| | | | RTLERLERDLPPEVL | dSLlRAIeEAerTLeRLerDLppeVLerFrpHLrLeIlLkKd |
| | | | ERFRPHLRLEILLKK | IkperprPpIrLeVlIeAdLsDpdSLlRAIeEAerTLeRLer |
| | | | DIKPE (SEQ ID | DLppeVLerFrpHLrLeIlLkKdIkperprPpIrLeVlIeAd |
| | | | NO: 27) | LsDpdSLlRAIeEAerTLeRLerDLppeVLerFrpHLrLeIl |
| | | | | LkKdIkperprPpIrLeVlIeAdLsDpdSLlRAIeEAerTLe |
| | | | | RLerDLppeVLerFrpHLrLeIlLkKdIkpe (SEQ ID |
| | | | | NO: 60) |
| HALC5_ | C5 | C5 | MDPKELEREALKNII | MdpkeLereALkNIIkLPkLIqdFKdSVmkELnKIIeLLeeR |
| 176 | | | KLPKLIQDFKDSVMK | RrEIDePLlpIIrKLQeeLqkkeMdpkeLereALkNIIkLPk |
| | | | ELNKIIELLEERRRE | LIqdFKdSVmkELnKIIeLLeeRRrEIDePLlpIIrKLQeeL |
| | | | IDEPLLPIIRKLQEE | qkkeMdpkeLereALkNIIkLPkLIqdFKdSVmkELnKIIeL |
| | | | LQKKE (SEQ ID | LeeRRrEIDePLlpIIrKLQeeLqkkeMdpkeLereALkNIi |
| | | | NO: 28) | kLPkLIqdFKdSVmkELnKIIeLLeeRRrEIDePLlpIIrKL |
| | | | | QeeLqkkeMdpkeLereALkNIikLPkLIqdFKdSVmkELnK |
| | | | | IIeLLeeRRrEIDePLlpIIrKLQeeLqkke (SEQ ID |
| | | | | NO: 61) |
| HALC6_ | C6 | C6 | RKIPYDPNRDLYITI | rkIpYDpnRDLYItItLtVrnnPdqkSFlqSIdLLikLLeQG |
| 208 | | | TLTVRNNPDQKSFLQ | YrVtInLvdFntKeEKeqALqQLrkIpYDpnRDLYItItLtV |
| | | | SIDLLIKLLEQGYRV | rnnPdqkSFlqSIdLLikLLeQGYrVtInLvdFntKeEKeqA |
| | | | TINLVDFNTKEEKEQ | LqQLrkIpYDpnRDLYItItLtVrnnPdqkSFlqSIdLLikL |
| | | | ALQQL (SEQ ID | LeQGYrVtInLvdFntKeEKeqALqQLRkIpYDpnRDLYItI |
| | | | NO: 29) | tLtVrnnPdqkSFlqSIdLLikLLeQGYrVtInLvdFntKeE |
| | | | | KeqALqQLRkIpYDpnRDLYItItLtVrnnPdqkSFlqSIdL |
| | | | | LikLLeQGYrVtInLvdFntKeEKeqALqQLrkIpYDpnRDL |
| | | | | YItItLtVrnnPdqkSFlqSIdLLikLLeQGYrVtInLvdFn |
| | | | | tKeEKeqALqQL (SEQ ID NO: 62) |
| HALC15- | C15 | C5 | DVPLTDPKNLNEFLY | dVpLtDPkNLNEFLyALGEGLkGMkNLkkLtLtFPSNPLTIp |
| 5_262 | | | ALGEGLKGMKNLKKL | GdIseGFrELGeGLkGMkNLeeLtVtFNdVpLtDPkNLnEFL |
| | | | TLTFPSNPLTIPGDI | yALGEGLkGMkNLkkLtLtFPSNPLTIpGdIseGFrELGeGL |
| | | | SEGFRELGEGLKGMK | kGMkNLeeLtVtFNdVpLtDPkNLNEFLyALGEGLkGMkNLk |
| | | | NLEELTVTFNDVPLT | kLtLtFPSNPLTIpGdIseGFrELGeGLkGMkNLeeLtVtFN |
| | | | DPKNLNEFLYALGEG | dVpLtDPkNLNEFLyALGEGLkGMkNLkkLtLtFPSNPLTIp |
| | | | LKGMKNLKKLTLTFP | GdIseGFrELGeGLkGMkNLeeLtVtFNdVpLtDPkNLNEFL |
| | | | SNPLTIPGDISEGFR | yALGEGLkGMkNLkkLtLtFPSNPLTIpGdIseGFrELGeGL |
| | | | ELGEGLKGMKNLEEL | kGMkNLeeLtVtFNdVpLtDPkNLNEFLyALGEGLkGMkNLk |
| | | | TVTFNDVPLTDPKNL | kLtLtFPSNPLTIpGdIseGFrELGeGLkGMkNLeeLtVtFN |
| | | | NEFLYALGEGLKGMK | dVpLtDPkNLNEFLyALGEGLkGMkNLkkLtLtFPSNPLTIp |
| | | | NLKKLTLTFPSNPLT | GdIseGFrELGeGLkGMkNLeeLtVtFNdVpLtDPkNLNEFL |
| | | | IPGDISEGFRELGEG | yALGEGLkGMkNLkkLtLtFPSNPLTIpGdIseGFrELGeGL |
| | | | LKGMKNLEELTVTFN | kGMkNLeeLtVtFNdVpLtDPkNLNEFLyALGEGLkGMkNLk |
| | | | (SEQ ID NO: 30) | kLtLtFPSNPLTIpGdIseGFrELGeGLkGMkNLeeLtVtFN |
| | | | | dVpLtDPkNLNEFLyALGEGLkGMkNLkkLtLtFPSNPLTIp |
| | | | | GdIseGFrELGeGLkGMkNLeeLtVtFNdVpLtDPkNLNEFL |
| | | | | yALGEGLkGMkNLkkLtLtFPSNPLtIpGdIseGFrELGeGL |
| | | | | kGMkNLeeLtVtFNdVpLtDPkNLNEFLyALGEGLkGMkNLk |
| | | | | kLtLtFPSNPLTIpGdIseGFrELGeGLkGMkNLeeLtVtFN |
| | | | | dVpLtDPkNLNEFLyALGEGLkGMkNLkkLtLtFPSNPLTIp |
| | | | | GdIseGFrELGeGLkGMkNLeeLtVtFNdVpLtDPkNLNEFL |
| | | | | yALGEGLkGMkNLkkLtLtFPSNPLTIpGdIseGFrELGeGL |
| | | | | kGMkNLeeLtVtFNdVpLtDPkNLNEFLyALGEGLkGMkNLk |
| | | | | kLtLtFPSNPLTIpGdIseGFrELGeGLkGMkNLeeLtVtFN |
| | | | | (SEQ ID NO: 63) |
| HALC18- | C18 | C6 | NIKIPNPKDLSELLK | nIKIPNPKDLSELLKKLGEGLkGLpNLktLtLtLsnIeLPed |
| 6_265 | | | KLGEGLKGLPNLKTL | AdLspGAeGLGeGLkGLpNLetLtFtIsnIkIPNPkDLSELL |
| | | | TLTLSNIELPEDADL | kKLGeGLkGLpNLktLtLtLsnIeLPedAdLspGAeGLGeGL |
| | | | SPGAEGLGEGLKGLP | kGLpNLetLtFtIsnIkIPNPkDLSELLkKLGeGLkGLpNLk |
| | | | NLETLTFTISNIKIP | tLtLtLsnIeLPedAdLspGAeGLGeGLkGLpNLetLtFtIs |
| | | | NPKDLSELLKKLGEG | nIKIPNPKDLSELLkKLGeGLkGLpNLktLtLtLsnIeLPed |
| | | | LKGLPNLKTLTLTLS | AdLspGAeGLGeGLkGLpNLetLtFtIsnIkIPNPkDLSELL |
| | | | NIELPEDADLSPGAE | kKLGeGLkGLpNLktLtLtLsnIeLPedAdLspGAeGLGeGL |
| | | | GLGEGLKGLPNLETL | kGLpNLetLtFtIsnIkIPNPkDLSELLkKLGeGLkGLpNLk |
| | | | TFTISNIKIPNPKDL | tLtLtlsnIeLPedAdLspGAeGLGeGLkGLpNLetLtFtIs |
| | | | SELLKKLGEGLKGLP | nIKIPNPKDLSELLkKLGeGLkGLpNLktLtLtLsnIeLPed |
| | | | NLKTLTLTLSNIELP | AdLspGAeGLGeGLkGLpNLetLtFtIsnIkIPNPkDLSELL |
| | | | EDADLSPGAEGLGEG | kKLGeGLkGLpNLktLtLtlsnIeLPedAdLspGAeGLGeGL |
| | | | LKGLPNLETLTFTIS | kGLpNLetLtFtIsnIkIPNPkDLSELLkKLGeGLkGLpNLk |
| | | | (SEQ ID NO: 31) | tLtLtlsnIeLPedAdLspGAeGLGeGLkGLpNLetLtFtIs |
| | | | | nIKIPNPKDLSELLKKLGEGLkGLpNLktLtLtLsnIeLPed |
| | | | | AdLspGAeGLGeGLkGLpNLetLtFtIsnIkIPNPkDLSELL |
| | | | | kKLGEGLkGLpNLktLtLtLsnIeLPedAdLspGAeGLGeGL |
| | | | | kGLpNLetLtFtIsnIkIPNPkDLSELLKKLGEGLkGLpNLk |
| | | | | tLtLtLsnIeLPedAdLspGAeGLGeGLkGLpNLetLtFtIs |
| | | | | nIKIPNPKDLSELLKKLGeGLkGLpNLktLtLtLsnIeLPed |
| | | | | AdLspGAeGLGeGLkGLpNLetLtFtIsnIkIPNPkDLSELL |
| | | | | kKLGeGLkGLpNLktLtLtLsnIeLPedAdLspGAeGLGeGL |
| | | | | kGLpNLetLtFtIsnIkIPNPkDLSELLkKLGeGLkGLpNLk |
| | | | | tLtLtlsnIeLPedAdLspGAeGLGeGLkGLpNLetLtFtIs |
| | | | | nIKIPNPKDLSELLKKLGEGLkGLpNLktLtLtLsnIeLPed |
| | | | | AdLspGAeGLGeGLkGLpNLetLtFtIsnIkIPNPkDLSELL |
| | | | | kKLGeGLkGLpNLktLtLtLsnIeLPedAdLspGAeGLGeGL |
| | | | | kGLpNLetLtFtIsnIkIPNPkDLSELLKKLGEGLkGLpNLk |
| | | | | tLtLtLsnIeLPedAdLspGAeGLGeGLkGLpNLetLtFtIs |
| | | | | (SEQ ID NO: 64) |
| HALC33- | C33 | C3 | PSNWPEVAKYFDLGK | PsNWpeVAkYfdLgKALkPIGeGLqNLkNLkhLdLsFsFsle |
| 3_343 | | | ALKPIGEGLQNLKNL | LYpGLPsNWpeVAkYFdLgKALkPIGeGLqNLkNLkhLdLsF |
| | | | KHLDLSFSFSLELYP | sFsLeLypgLPsNWpeVAkYFdLgKALkPIGeGLqNLkNLkh |
| | | | GLPSNWPEVAKYFDL | LdLsFsFsLeLYpGLPsNWpeVAkYFdLgKALkPIGeGLqNL |
| | | | GKALKPIGEGLQNLK | kNLkhLdLsFsFsLeLYpGLPsNWpeVAkYFdLgKALkPIGe |
| | | | NLKHLDLSFSFSLEL | GLqNLkNLkhLdLsFsFsLeLYpGLPsNWpeVAkYFdLgKAL |
| | | | YPGLPSNWPEVAKYF | kPIGeGLqNLkNLkhLdLsFsFsLeLYpgLPsNWpeVAkYFd |
| | | | DLGKALKPIGEGLQN | LgKALKPIGeGLqNLkNLkhLdLsFsFsLeLYpgLPsNWpeV |
| | | | LKNLKHLDLSFSFSL | AkYFdLgKALkPIGeGLqNLkNLkhLdLsFsFsLeLYPGlPs |
| | | | ELYPGLPSNWPEVAK | NWpEVAkYFdLgKALkPIGeGLqNLkNLkhLdLsFsFsLeLY |
| | | | YFDLGKALKPIGEGL | PGlPsNWpeVAkYFdLgKALkPIGeGLqNLkNLkhLdLsFsF |
| | | | QNLKNLKHLDLSFSF | sLeLYPGlPsNWpeVAkYFdLgKALkPIGeGLqNLkNLkhLd |
| | | | SLELYPGLPSNWPEV | LsFsFsLeLyPGlPsNWpEVAkYFdLGKALKPIGeGLqNLkN |
| | | | AKYFDLGKALKPIGE | LkhLdLsFsFsLeLYPglPsNWpEVAkYFdLgKALkPIGeGL |
| | | | GLQNLKNLKHLDLSF | qNLkNLkhLdLsFsFsLeLyPGlPsNWpeVAkYFdLGKALKP |
| | | | SFSLELYPGLPSNWP | IGeGLqNLkNLkhLdLsFsFsLeLyPGlPsNWpeVAkYFdLg |
| | | | EVAKYFDLGKALKPI | KALKPIGeGLqNLkNLkhLdLsFsFsLeLypGlPsNWpeVAk |
| | | | GEGLQNLKNLKHLDL | YFdLGKALKPIGeGLqNLkNLkhLdLsFsFsLeLyPGlPsNW |
| | | | SFSFSLELYPGLPSN | peVAkYFdLGKALKPIGeGLqNLkNLkhLdLsFsFsLeLypG |
| | | | WPEVAKYFDLGKALK | lPsNWpeVAkYFdLGKALKPIGeGLqNLkNLkhLdLsFsFsL |
| | | | PIGEGLQNLKNLKHL | eLyPGlPsNWpeVAkYFdLGKALkPIGeGLqNLkNLkhLdLs |
| | | | DLSFSFSLELYPGLP | FsFsLeLyPGlPsNWpeVAkYFdLGKALkPIGeGLqNLkNLk |
| | | | SNWPEVAKYFDLGKA | hLdLsFsFsLeLyPGlPsNWpeVAkYFdLGKALkPIGeGLqN |
| | | | LKPIGEGLQNLKNLK | LkNLkhLdLsFsFsLeLyPGlPsNWpeVAkYFdLGKALKPIG |
| | | | HLDLSFSFSLELYPG | eGLqNLkNLkhLdLsFsFsLeLyPGlPsNWpEVAkYFdLGKA |
| | | | LPSNWPEVAKYFDLG | LkPIGeGLqNLkNLkhLdLsFsFsLeLYPGlPsNWpEVAkYF |
| | | | KALKPIGEGLQNLKN | dLgKALkPIGeGLqNLkNLkhLdLsFsFsLeLypGlPsNWpe |
| | | | LKHLDLSFSFSLELY | VAkYFdLGKALKPIGeGLqNLkNLkhLdLsFsFsLeLyPGlP |
| | | | PGLPSNWPEVAKYFD | sNWpeVAkYFdLgKALkPIGeGLqNLkNLkhLdLsFsFsLeL |
| | | | LGKALKPIGEGLQNL | ypGlPsNWpeVAkYFdLGKALKPIGeGLqNLkNLkhLdLsFs |
| | | | KNLKHLDLSFSFSLE | FsLeLyPGlPsNWpeVAkYFdLgKALkPIGeGLqNLkNLkhL |
| | | | LYPGLPSNWPEVAKY | dLsFsFsLeLyPGlPsNWpeVAkYFdLGKALkPIGeGLqNLk |
| | | | FDLGKALKPIGEGLQ | NLkhLdLsFsFsLeLyPGlPsNWpeVAkYFdLgKALkPIGeG |
| | | | NLKNLKHLDLSFSFS | LqNLkNLkhLdLsFsFsLeLyPGlPsNWpeVAkYFdLGKALk |
| | | | LELYPGL | PIGeGLqNLkNLkhLdLsFsFsLeLyPGlPsNWpeVAkYFdL |
| | | | (SEQ ID NO: 32) | GKALKPIGeGLqNLkNLkhLdLsFsFsLeLyPGlPsNWpeVA |
| | | | | kYFdLGKALkPIGeGLqNLkNLkhLdLsFsFsLelyPGl |
| | | | | (SEQ ID NO: 65) |
| HALC6_ | C6 | C6 | PPIPPPSFKLEISPA | ppiPppsFkLeISpAFLELVqLVIdLHpndeeVrkeLIeNLI |
| 220 | | | FLELVQLVIDLHPND | sRIgKSDNVppetIsLdISeAALELFeWIfeKFpdDedVHrr |
| | | | EEVRKELIENLISRI | LIeSFInKRkFsssspLdTPsLdISeRFIeLVkyILeKYpeD |
| | | | GKSDNVPPETISLDI | eeIKqKLidSLlNLLGSYppiPppsFkLeISpAFLELVqLVI |
| | | | SEAALELFEWIFEKF | dLHpndeeVrkeLIeNLIsRIgKSDNVppetIsLdISeAALE |
| | | | PDDEDVHRRLIESFI | LFeWIfeKFpdDedVHrrLIeSFInKRkFsssspLdTpsLdI |
| | | | NKRKFSSSSPLDTPS | SeRFIeLVkyILeKYpeDeeIKqKLidSLlNLLgSYppiPpp |
| | | | LDISERFIELVKYIL | sFkLeISpAFLELVqLVIdLHpndeeVrkeLIeNLIsRIgKS |
| | | | EKYPEDEEIKQKLID | DNVppetIsLdISeAALELFeWIfeKFpdDedVHrrLIeSFI |
| | | | SLLNLLGSY | nKRkFsssspLdTPsLdISeRFIeLVkyILekYpeDeeIKqK |
| | | | (SEQ ID NO: 33) | LidSLlNLLGSYppiPppsFkLeISpAFLELVqLVIdLHpnd |
| | | | | eeVrkeLIeNLIsRIgKSDNVppetIsLdISeAALELFeWIF |
| | | | | eKFpdDedVHrrLIeSFInKRkFsssspLdTpsLdISeRFIe |
| | | | | LVkyILeKYpeDeeIKqKLidSLlNLLGSYppiPppsFkLeI |
| | | | | SpAFLELVqLVIdLHpndeeVrkeLIeNLIsRIgKSDNVppe |
| | | | | tIsLdISeAALELFeWIFeKFpdDedVHrrLIeSFInKRkFs |
| | | | | ssspLdTpsLdISeRFIeLVkYILeKYpeDeeIKqKLidSLl |
| | | | | NLLGSYppiPppsFkLeISpAFLELVqLVIdLHpndeeVrke |
| | | | | LIeNLIsRIgKSDNVppetIsLdISeAALELFeWIFeKFpdD |
| | | | | edVHrrLIeSFInKRkFsssspLdTPsLdISeRFIeLVkYIL |
| | | | | eKYpeDeeIKqKLidSLlNLLGSY |
| | | | | (SEQ ID NO: 66) |
| HALC24- | C24 | C6 | SKEKLGIQQDLFEGI | sKeKLGIqqdLFegIIaTLLsHkDprVLyLMVTILkLTGssP |
| 6_316 | | | IATLLSHKDPRVLYL | sKeKLGIqqdLFegIIaTLLsHkDprVLyLMVTILkLTGssP |
| | | | MVTILKLTGSSPSKE | sKeKLGIqqdLFegIIaTLLsHkDprVLyLMVTILkLTGssP |
| | | | KLGIQQDLFEGIIAT | skeKLGIqqdLFegIIaTLLsHkDprVLyLMVTILkLTGssp |
| | | | LLSHKDPRVLYLMVT | sKeKLGIqqdLFegIIaTLLsHkDprVLyLMVTILkLTGssP |
| | | | ILKLTGSSPSKEKLG | sKeKLGIqqdLFegIIaTLLsHkdprVLyLMVTILkLTGssP |
| | | | IQQDLFEGIIATLLS | sKeKLGIqqdLFegIIaTLLsHkdprVLyLMVTILkLTGssP |
| | | | HKDPRVLYLMVTILK | skeKLGIqqdLFegIIaTLLsHkdprVLyLMVTILkLTGssp |
| | | | LTGSSPSKEKLGIQQ | sKeKLGIqqdLFegIIaTLLsHkdprVLyLMVTILkLTGssP |
| | | | DLFEGIIATLLSHKD | sKeKLGIqqdLFegIIaTLLsHkdprVLyLMVTILkLTGssP |
| | | | PRVLYLMVTILKLTG | sKeKLGIqqdLFegIIaTLLsHkdprVLyLMVTILkLTGssP |
| | | | SSP | skeKLGIqqdLFegIIaTLLsHkDprVLyLMVTILkLTGssp |
| | | | (SEQ ID NO: 34) | sKeKLGIqqdLFegIIaTLLsHkDprVLyLMVTILkLTGssP |
| | | | | sKeKLGIqqdLFegIIaTLLsHkdprVLyLMVTILkLTGssP |
| | | | | sKeKLGIqqdLFegIIaTLLsHkdprVLyLMVTILkLTGssP |
| | | | | skeKLGIqqdLFegIIaTLLsHkdprVLyLMVTILkLTGssp |
| | | | | sKeKLGIqqdLFegIIaTLLsHkdprVLyLMVTILkLTGssP |
| | | | | sKeKLGIqqdLFegIIaTLLsHkdprVLyLMVTILkLTGssP |
| | | | | sKeKLGIqqdLFegIIaTLLsHkdprVLyLMVTILkLTGssP |
| | | | | skeKLGIqqdLFegIIaTLLsHkDprVLyLMVTILkLTGssp |
| | | | | sKeKLGIqqdLFegIIaTLLsHkDprVLyLMVTILkLTGssP |
| | | | | sKeKLGIqqdLFegIIaTLLsHkDprVLyLMVTILkLTGssP |
| | | | | sKeKLGIqqdLFegIIaTLLsHkDprVLyLMVTILkLTGssP |
| | | | | skeKLGIqqdLFegIIaTLLsHkDprVLyLMVTILkLTGssp |
| | | | | (SEQ ID NO: 67) |
| HALC20- | C20 | C5 | SLGWKVLLHLDLTWY | SlgWkVlLhLdLtWyPGadYtIdHdDMtrAArALAhGFErAA |
| 5_308 | | | PGADYTIDHDDMTRA | hSFAeAIGsTgSlgWkVlLhLdLtWyPGadYtIdHdDMtrAA |
| | | | ARALAHGFERAAHSF | rALAhGFErAAhSFAeAIGsTgSlgWkVlLhLdLtWyPGadY |
| | | | AEAIGSTGSLGWKVL | tIdHdDMtRAArALAhGFERAAhSFAeAIGsTgSlgWkVlLh |
| | | | LHLDLTWYPGADYTI | LdLtWyPGadYtIdHdDMTRAArALAhGFERAAhSFAeAIGS |
| | | | DHDDMTRAARALAHG | TgSlgWkVlLhLdLtWyPGadYtIdHdDMTRAArALAhGFER |
| | | | FERAAHSFAEAIGST | AAhSFAeAIGsTgSlgWkVlLhLdLtWyPGadYtIdHdDMTR |
| | | | GSLGWKVLLHLDLTW | AArALAhGFERAAhSFAeAIGsTgSlgWkVlLhLdLtWyPGa |
| | | | YPGADYTIDHDDMTR | dYtIdHdDMTRAArALAhGFERAAhSFAeAIGsTgSlgWkVl |
| | | | AARALAHGFERAAHS | LhLdLtWyPGadYtIdHdDMTRAArALAhGFERAAhSFAeAI |
| | | | FAEAIGSTGSLGWKV | GSTgSlgWkVlLhLdLtWyPGadYtIdhdDMtRAArALAhGF |
| | | | LLHLDLTWYPGADYT | ERAAhSFAeAIGSTgSlgWkVlLhLdLtWyPGadYtIdhdDM |
| | | | IDHDDMTRAARALAH | tRAArALAhGFERAAhSFAeAIgSTgSlgWkVlLhLdLtWyP |
| | | | GFERAAHSFAEAIGS | GadYtIdhdDMtRAArALAhGFERAAhSFAeAIGSTgSlgWk |
| | | | TG | VlLhLdLtWyPGadYtIdHdDMtRAArALAhGFERAAhSFAe |
| | | | (SEQ ID NO: 35) | AIGSTgSlgWkVlLhLdLtWyPGadYtIdHdDMTRAArALAh |
| | | | | GFERAAhSFAeAIGsTgSlgWkVlLhLdLtWyPGadYtIdHd |
| | | | | DMTRAArALAhGFERAAhSFAeAIGsTgSlgWkVlLhLdLtW |
| | | | | yPGadYtIdHdDMTRAArALAhGFERAAhSFAeAIGsTgSlg |
| | | | | WkVlLhLdLtWyPGadYtIdHdDMTRAArALAhGFERAAhSF |
| | | | | AeAIGsTgSlgWkVlLhLdLtWyPGadYtIdHdDMTRAArAL |
| | | | | AhGFERAAhSFAeAIGsTgSlgWkVlLhLdLtWyPGadYtId |
| | | | | HdDMTRAArALAhGFERAAhSFAeAIGsTgSlgWkVlLhLdL |
| | | | | tWyPGadYtIdHdDMTrAArALAhGFERAAhSFAeAIGsTgS |
| | | | | lgWkVlLhLdLtWyPGadYtIdHdDMTrAArALAhGFErAAh |
| | | | | SFAeAIGsTg |
| | | | | (SEQ ID NO: 68) |
| HALC25- | C25 | C5 | EGAAIAENLATAYQG | EGaaIAeNLAtAYQGIGeTLpSLqDLrvLhLsViFsAEGsSp |
| 5_341 | | | IGETLPSLQDLRVLH | EGAaIAeNLAtAYQGIGeTLpSLqDLrvLhLsViFSAeGsSp |
| | | | LSVIFSAEGSSPEGA | EGAAIAeNLAtAYQGIGeTLpSLqdLrvLhLsViFSAeGSSp |
| | | | AIAENLATAYQGIGE | EGAAIAeNLAtAYQGIGeTLpSLqdLrvLhLsViFSAEGSSp |
| | | | TLPSLQDLRVLHLSV | EGAAIAeNLAtAYQGIGeTLpSLqdLrvLhLsViFsAeGssp |
| | | | IFSAEGSSPEGAAIA | EGaAIAeNLAtAYQGIGeTLpSLqdLrvLhLsViFsAEGsSp |
| | | | ENLATAYQGIGETLP | EGAAIAeNLAtAYQGIGeTLpSLqdLrvLhLsViFSAeGsSp |
| | | | SLQDLRVLHLSVIFS | EGAAIAeNLAtAYQGIGeTLpSLqDLrvLhLsViFsAeGsSp |
| | | | AEGSSPEGAAIAENL | EGAAIAeNLAtAYQGIGeTLpSLqDLrvLhLsViFsAeGSSp |
| | | | ATAYQGIGETLPSLQ | EGaAIAeNLAtAYQGIGeTLpSLqDLrvLhLsViFsAeGssp |
| | | | DLRVLHLSVIFSAEG | EGaaIAeNLAtAYqGIGeTLpSLqDLrvLhLsViFsAeGsSp |
| | | | SSPEGAAIAENLATA | EGAaIAeNLAtAYqGIGeTLpSLqDLrvLhLsViFsAeGsSp |
| | | | YQGIGETLPSLQDLR | EGAaIAeNLAtAYqGIGeTLpSLqDLrvLhLsViFsAeGsSp |
| | | | VLHLSVIFSAEGSSP | EGAaIAeNLAtAYQGIGeTLpSLqDLrvLhLsViFsAeGSSp |
| | | | (SEQ ID NO: 36) | EGAaIAeNLAtAYQGIGeTLpSLqdLrvLhLsViFsAeGssp |
| | | | | EGAaIAeNLAtAYQGIGeTLpSLqdLrvLhLsViFSAEGsSp |
| | | | | EGAaIAeNLAtAYQGIGeTLpSLqdLrvLhLsViFSAeGsSp |
| | | | | EGAAIAeNLAtAYQGIGeTLpSLqdLrvLhLsViFSAeGsSp |
| | | | | EGAAIAeNLAtAYQGIGeTLpSLqdLrvLhLsViFsAeGSSp |
| | | | | EGaAIAeNLAtAYqGIGeTLpSLqdLrvLhLsViFsAeGssp |
| | | | | EGaAIAeNLAtAYqGIGeTLpSLqDLrvLhLsViFsAeGsSp |
| | | | | EGAaIAeNLAtAYqGIGeTLpSLqDLrvLhLsViFsAeGsSp |
| | | | | EGAAIAeNLAtAYqGIGeTLpSLqDLrvLhLsViFsAeGsSp |
| | | | | EGAAIAeNLAtAYQGIGeTLpSLqDLrvLhLsViFsAeGSSp |
| | | | | EGAAIAeNLAtAYQGIGeTLpSLqDLrvLhLsViFsAeGssp |
| | | | | (SEQ ID NO: 69) |
| HALC18- | C18 | C6 | GERTDNPYYIGLLLK | GerTdNPYYIGLLLKHLGEGLkKNkKLekLkLdLPVFttepN |
| 6_278 | | | HLGEGLKKNKKLEKL | pILeeGFkLLGeGLANIeSpLdLeIkILGerTdNPYYIGLLL |
| | | | KLDLPVFTTEPNPIL | KHLGEGLKKNkKLekLkLdLPVFttepNpILeEGFkLLGEGL |
| | | | EEGFKLLGEGLANIE | ANIeSpLdLeikILGerTdNPYYIGLLLKHIGEGLkKNkKLe |
| | | | SPLDLEIKILGERTD | kLkldLPVFtTepNpILeEGFkLLGEGLANIeSpLdleikIL |
| | | | NPYYIGLLLKHLGEG | GerTdNPYYIGLLLKHIGEGLkKNkKLeklkldlPVFttepN |
| | | | LKKNKKLEKLKLDLP | piLeeGFKLLGEGLaNIeSpLdleikILGerTdNPYYIGLIL |
| | | | VFTTEPNPILEEGFK | KHIGEGLkKNkKLeklklDIPVFttePNpiLEeGFKLLGEGL |
| | | | LLGEGLANIESPLDL | aNIeSpLdleikILGerTdNPYYIGLLLKHLGEGLkKNkKLe |
| | | | EIKILGERTDNPYYI | KLklDLPVFtTePNpILEEGFKLLgEGLANIeSpLdLeIkIL |
| | | | GLLLKHLGEGLKKNK | GeRTdNPYYIGLLLKHLGEGLKKNKKLeKLkLdLPVFTTePN |
| | | | KLEKLKLDLPVFTTE | PILEeGFkLLGeGLANIeSpLdLeIkILGerTdNPyYIGLLL |
| | | | PNPILEEGFKLLGEG | KHLGEGLKKNkKLekLkLdLPVFTTePNPILEeGFKILGeGL |
| | | | LANIESPLDLEIKIL | ANIeSpLdLeIkILGerTdNPyYIGLLLKHLGEGLKKNkKLe |
| | | | (SEQ ID NO: 37) | KLkLdLPVFTtePNpILEeGFKLLGEGLAnIeSpLdLeIkIL |
| | | | | GerTdNPYYIGLLLKHLGEGLKKNkKLekLkLdLPVFtTepN |
| | | | | pILEeGFkLLGeGLANIeSpLdLeIkILGerTdNPYYIGLLL |
| | | | | KHLGeGLKKNkKLekLkLdLPVFtTepNpILeeGFkLLGeGL |
| | | | | ANIeSpLdLeIkILGerTdNPyYIGLLLKHLGeGLkkNkKLe |
| | | | | kLkldLPVFtTepNpILeeGFkLLGeGLaNIeSpLdLeIkIL |
| | | | | GerTdNPyYIGLLLKHLGeGLkkNkKLekLkldLPVFtTepN |
| | | | | pILeeGFkLlGeGLaNIeSpLdleIkILGerTdNPyYIGlLL |
| | | | | kHlGeGLkkNkKLekLkldlPvftTepNpILeeGfkLlGeGl |
| | | | | anIeSpldleikILGerTdnPyYIGllLkhlGeGLkknkkle |
| | | | | klkldlPVFttepNpILeeGFkLlGeGLaniespldleikIL |
| | | | | GerTdnPyYIGLILkHlGeGLkknkkLeklkldlPVFtTepN |
| | | | | pILeeGFkLLGeGLanIeSpLdleikILGerTdNPyYIGLIL |
| | | | | kHLGeGLkkNkKLekLkldLPVFtTepNpILeeGFkLLGeGL |
| | | | | aNIeSpLdLeIkILGerTdNPYYIGLLLKHLGeGLkKNkKLe |
| | | | | kLkLdLPVFtTepNpILeeGFkLLGeGLaNIeSpLdLeIkIL |
| | | | | (SEQ ID NO: 70) |
| HALC42- | C42 | C7 | PSLTLNDFGDLGKGL | PsLtLnDFgDLGkGLGeGLqGMeNLeKLQLTITLkLtVsTps |
| 7_351 | | | GEGLQGMENLEKLQL | LtLnDFgDLGkGLGEGLqGMeNLeKLQLTITLkLtVsTpsLt |
| | | | TITLKLTVSTPSLTL | LnDFgDLGkGLGEGLqGMeNLeKLQLTITLkLtVsTpsLtLn |
| | | | NDFGDLGKGLGEGLQ | DFgDLGkGLgEGLQGMeNLeKLQLTITLkltvSTpsLtLnDF |
| | | | GMENLEKLQLTITLK | gDLGKGLgEGLQGMEnLeKLQlTiTlklTvstpsltInDFGD |
| | | | LTVSTPSLTLNDFGD | LGKGLGEGLQGMEnLekLQlTiTlKITvstPSLTINDFGDLG |
| | | | LGKGLGEGLQGMENL | KGLGEGLQGMEnLeKLQLTiTlKlTvStpslTINDFGDLGKG |
| | | | EKLQLTITLKLTVST | LGEGLQGMenLeKLQLTiTlKITvStpSLTLNdFGdLGKgLG |
| | | | PSLTLNDFGDLGKGL | EGLQGMenLeKLQlTiTlKITvStpslTLndFGdLGkgLGeG |
| | | | GEGLQGMENLEKLQL | LQgMenLeKLQLTITlKITvStpsLtLndFGdLGkGLGeGLQ |
| | | | TITLKLTVSTPSLTL | gMenLEKLQLTITLKLTVStpsLtLndFGdLGkGLGeGLQGM |
| | | | NDFGDLGKGLGEGLQ | eNLEKLQLTITLKLTVsTPsLtLndFGdLGkGLGEGLQGMeN |
| | | | GMENLEKLQLTITLK | LEKLQLTITLKLTVsTpsLtLndFGDLGKGLGEGLQGMeNLE |
| | | | LTVSTPSLTLNDFGD | KLQLTITLKLTVSTpsLtLnDFgDLGKGLGEGLQGMeNLEKL |
| | | | LGKGLGEGLQGMENL | QLTITLKLtVsTpsLtLnDFgDLGKGLGEGLQGMeNLEKLQL |
| | | | EKLQLTITLKLTVST | TITLkLtVsTpsLtLnDFGDLGKGLGEGLQGMeNLEKLQLTI |
| | | | (SEQ ID NO: 38) | tLkLtVsTpsLtLnDFGDLGKGLGEGLQGMeNLEKLQLTItL |
| | | | | kLtVsTPsLtLnDFGDLGKGLGEGLQGMENLEKLQLtItLkL |
| | | | | tVsTpsLtLNDFGDLGKGLGEGLQGMENLEKLqLtItLkLtV |
| | | | | sTpsLTLNDFGDLGKGLGEGLQGMENLEKLqLtItLkLtVsT |
| | | | | psLTLNDFGDLGKGLGEGLQGMENLEKLqLtItLkLtVsTps |
| | | | | LTLNDFGDLGKGLGEGLQGMENLEKLqLtItLkLtVsTpsLT |
| | | | | LNDFGDLGKGLGEGLQGMENLEKLQLTItLkLtVsTPsLtLn |
| | | | | dFGDLGKGLGEGLQGMENLEKLQLTITLkLtVsTpsLtLndF |
| | | | | GdLGKGLGEGLQGMENLEKLQLTITLkLtVsTpsLtLndFGd |
| | | | | LGkGLGeGLQGMeNLEKLQLTITLKLtVsTpsLtLndFGdLG |
| | | | | kGLGeGLqgMenLeKLQLTITLKLtVsTpsLtLndFgDLGKG |
| | | | | LGeGLqGMenLeKLQLTITLkLtVsTpsLtLndFgDLGkGLG |
| | | | | eGLqGMeNLEKLQLTITLkLtVsTPsLtLndFgDLGkGLGeG |
| | | | | LqGMeNLEKLQLTITLkLtVsTpsLtLndFgDLGkGLGEGLq |
| | | | | GMeNLEKLQLTItLkLtVsTpsLtLndFgDLGkGLGEGLqGM |
| | | | | eNLEKLQLTItLkLtVsTpsLtLndFgDLGkGLGEGLqGMeN |
| | | | | LeKLQLTItLkLtVsTpsLtLndFgDLGkGLGEGLqGMeNLe |
| | | | | KLQLTItLkLtVsTpsLtLnDFGDLGKGLGEGLqGMeNLeKL |
| | | | | QLTItLkLtVsTPsLtLnDFGDLGkGLGEGLQGMeNLeKLQL |
| | | | | TItLkLtVsTpsLtLnDFGDLGkGLGEGLQGMeNLeKLQLTI |
| | | | | tLkLtVsTpsLtLnDFGDLGKGLGEGLQGMeNLeKLQLTITL |
| | | | | kLtVsTpsLtLnDFGDLGKGLGEGLQGMeNLeKLQLTITLKL |
| | | | | tVsTpsLtLnDFGDLGkGLGEGLqGMeNLeKLQLTITLkLtV |
| | | | | sTpsLtLnDFGDLGkGLGeGLqGMeNLeKLQLTITLkLtVsT |
| | | | | (SEQ ID NO: 71) |

In some embodiments, any N-terminal methionine residue is deleted in the polypeptides of the disclosure. In other embodiments, any N-terminal methionine residue is present in the polypeptides of the disclosure. In some embodiments, the polypeptide is at least 75% identical to the reference sequence. In other embodiments, the polypeptide is at least 90% identical to the reference sequence. In further embodiments, the polypeptide is at least 95% identical to the reference sequence.

In some embodiments, at least 50%, 55%, 60%, 65%, 70%, 75%, 80%, 85%, 90%, 91%, 92%, 93%, 94%, 95%, 96%, 97%, 98%, 99%, or 100% of substitutions relative to the reference amino acid sequence are at surface residues as defined in Table 1. The positions of surface residues are shown in lower case in the sequences (SEQ ID NO:1-5 and 39-71) shown in the far right column of Table 1; these sequences include one or more chains of the sequence of SEQ ID NO:1-38, and thus one of skill in the art will readily understand where the surface residues are present in SEQ ID NO:1-38. Surface or solvent exposed residues are more adaptable to substitution, especially with similar charged or polar amino acids, as they contribute less to the overall stability and structure of the protein fold when compared to residues in the protein core.

In other embodiments, at least 50%, 55%, 60%, 65%, 70%, 75%, 80%, 85%, 90%, 91%, 92%, 93%, 94%, 95%, 96%, 97%, 98%, 99%, or 100% of core residues, as defined in Table 1 are maintained as in the reference amino acid sequence. . The positions of core residues are shown in upper case in the sequences (SEQ ID NO:1-5 and 39-71) shown in the far right column of Table 1; these sequences include one or more chains of the sequence of SEQ ID NO:1-38, and thus one of skill in the art will readily understand where the core residues are present in SEQ ID NO:1-38. Core or non-solvent exposed residues are less adaptable to substitution as they contribute more to the overall stability and structure of the protein fold when compared to residues on the protein surface that are solvent exposed. Core residues stabilize the protein through hydrophobic packing interactions, hydrogen bonding, and van der Waals interactions among other interactions.

In some embodiments, relative to the reference sequence are conservative amino acid substitutions. As used herein, a “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Particular conservative substitutions include, but are not limited to, Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, the polypeptides may further comprise one or more functional domains. The polypeptides may comprise any further functional domain fused to the polypeptide that may be of use for an intended purpose. In various non-limiting embodiments, the resulting fusion protein comprises an additional functional domain such as detectable proteins, purification tags, protein antigens, and protein therapeutics. The functional domain may be a genetic fusion or may be otherwise covalently linked to the polypeptide. In one embodiment, the disclosure provides fusion proteins comprising the polypeptide of any embodiment herein linked to a protein antigen. In this embodiment, the linkage may be direct, or the polypeptide and protein antigen may be separated by an amino acid linker. The linker may be of any suitable length and amino acid composition. In one embodiment, the linker is a flexible linker, including but not limited to a GlySer-rich linker, which may be of any suitable length, including but not limited to 3-40, 3-30, 3-25, 3-20, 3-15, and 3-10 amino acids in length. The protein antigen may be any antigen appropriate for an intended use. Non-limiting examples of such protein antigens include protein antigens, or antigenic fragments thereof, of viral and bacterial proteins, including but not limited to human immunodeficiency virus (HIV), coronavirus, and influenza antigens.

In another embodiment, the disclosure provides cyclic homo-oligomers, comprising one or a plurality of a polypeptide or fusion protein of any embodiment herein. The cyclic homo-oligomers may be used, for example, in small molecule binding and catalysis, as building blocks for nanocage assemblies, scaffolding of protein binders and building nanomaterials, and for scaffolding antigens for generating an immune response against the antigen. In some embodiments, the cyclic homo-oligomers comprise a plurality of identical polypeptides or fusion proteins of any embodiment herein.

In one embodiment, the cyclic homo-oligomer has a symmetry (“Sym”) as listed in Table 1. In other embodiments, the cyclic homo-oligomer has a pseudosymmetry (“P-Sym”; number of chains) as listed in Table 1. In further embodiments, the cyclic homo-oligomer comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from SEQ ID NO:1-5 and 39-71. These sequences are shown in Table 1.

As shown in the examples that follow, the cyclic homo-oligomers of the disclosure are very stable. In one embodiment, the cyclic homo-oligomer maintains its secondary structure at temperatures up to 95° C. In other embodiments, wherein the cyclic homo-oligomer has a size along its largest dimension of between about 5 and about 16 nm, or between about 7 and about 14 nm. As used herein, “about” means +/−5% of the recited value.

The disclosure provides nucleic acids encoding the polypeptide or fusion protein of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides and fusion proteins of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptides, fusion proteins, cyclic homo-oligomers, nucleic acids, and/or expression vectors (i.e.: episomal or chromosomally integrated), disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

The disclosure also provides methods for designing a polypeptide capable of forming a cyclic homo-oligomer, comprising any combination of steps as disclosed in the attached examples.

The disclosure further provides methods for use of a polypeptide, cyclic homo-oligomer, nucleic acid, expression vector, and/or host cell of any embodiment herein for any suitable purpose, including but not limited to small molecule binding and catalysis, as building blocks for nanocage assemblies, and for scaffolding antigens for generating an immune response against the antigen. In one embodiment, the disclosure provides methods for generating an immune response, comprising administering to a subject in need thereof a cyclic homo-oligomer comprising a fusion protein comprising a protein antigen of any embodiment herein, wherein the cyclic homo-oligomer comprises the protein antigen scaffolded on a surface of the cyclic homo-oligomer, in an amount effective to generate an immune response against the antigen in the subject.

In another embodiment, the disclosure provides methods for increasing binding of a binder to a therapeutically relevant target, comprising scaffolding the binder protein or molecule through a genetic fusion or chemical linkage to any embodiment herein. The oligomerization of the binder protein or molecule through using the oligomers herein will increase their avidity when exposed to a target, especially if that target is present in a cluster for example on the surface of a cell. The increased avidity through the oligomerization will allow for a slower dissociation rate from the target as multiple targets can be bound with the oligomer allowing for example to efficiently block and neutralize a surface receptor of a pathogen that binds to a host target.

### EXAMPLES

### Abstract

Deep learning generative approaches provide an opportunity to broadly explore protein structure space beyond the sequences and structures of natural proteins. Here we use deep network hallucination to generate a wide range of symmetric protein homo-oligomers given only a specification of the number of protomers and the protomer length. Crystal structures of 7 designs are very close to the computational models (median RMSD: 0.6 Å), as are 3 cryoEM structures of giant rings with up to 1550 residues, C33 symmetry, and 10 nanometer in diameter; all differ considerably from previously solved structures. Our results highlight the rich diversity of new protein structures that can be created using deep learning, and pave the way for the design of increasingly complex nanomachines and biomaterials.

Cyclic protein oligomers play key roles in almost all biological processes and have many applications, ranging from small molecule binding and catalysis to building blocks for nanocage assemblies, Current approaches to designing cyclic protein oligomers require specification of the structure of the protomers in advance, and with the exception of parametrically designed helical bundles, have involved rigid body docking of previously characterized monomers into higher order symmetric structures followed by interface optimization to confer low energy to the assembled state. The requirement that the protomer structure be specified in advance has limited exploration of the full space of oligomeric structures; in particular assemblies in which the chains are more intertwined. We reasoned that deep network hallucination could enable the design of higher-order protein assemblies in one step, without pre-specification or experimental confirmation of the structures of the protomers, provided that a suitable loss function could be formulated.

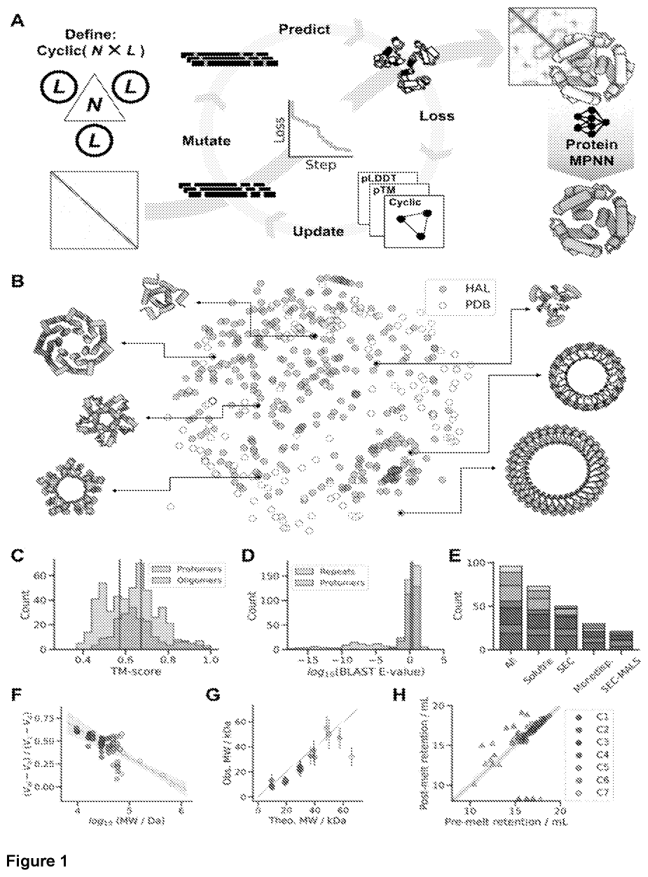

We set out to broadly explore the space of cyclic protein homo-oligomers by developing a method for hallucinating such structures that places no constraints on the structures of either the protomers or the overall assemblies. Starting from only a choice of chain length L and oligomer valency N (2 for a dimer, 3 for a trimer, etc.), the method initializes a random amino acid sequence to begin a Monte Carlo search in sequence space (FIG. 1A). The loss function guiding the search is computed by inputting N copies of the sequence into the AlphaFold2™ (AF2) network (25), and combining structure prediction confidence metrics (pLDDT and p™) with a measure of cyclic symmetry; the standard deviation of the distances between the center of mass of adjacent protomers within the predicted structure.

We found that monomers and dimeric to heptameric assemblies could readily be generated by this procedure for chains of 65 to 130 amino acids, with converging trajectories typically coalescing to cyclic homo-oligomeric structures within a few hundred steps (approximately one week of CPU-time). The resulting structures are topologically diverse, spanning all-α, mixed α/β and all-β structures and differ from structurally-verified cyclic de novo designs present in the PDB (FIG. 1B). These assemblies, which we term HALs, also differ from natural proteins, with the median closest relatives in the PDB having TM-scores of 0.67 and 0.57 for the protomers and oligomers respectively (29% of the structures have TM-scores<0.5, which constitutes the cutoff for fold assignment in CATH/SCOP (26) (FIG. 1C), and sequences unrelated to natural ones (FIG. 1D), indicating considerable generalization beyond the PDB training set.

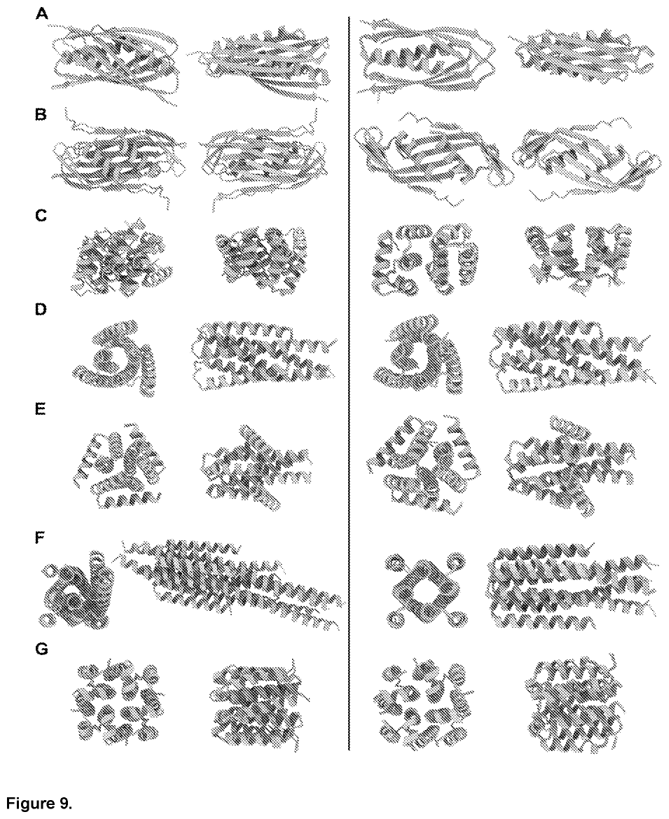

We selected 150 designs with pLDDT>0.7 and pTM>0.7 for experimental testing. However, virtually none showed significant soluble expression when produced in E. coli (median soluble yield: 9 mg per liter of culture-equivalent, FIG. 5), and of the few that were marginally soluble none had both the expected oligomerization state by size-exclusion chromatography (SEC) and a circular dichroism (CD) profile consistent with the hallucinated structure. We speculated that this failure could be a consequence of over-fitting during MCMC optimization leading to the generation of adversarial sequences. Analogous neural network activation maximization approaches with 2D images similarly can lead to non-viable solutions (27-29). To eliminate such over-fitting, we generated new sequences for the hallucinated oligomer backbones using the recently developed ProteinMPNN™ sequence design method. For each original backbone, 24 to 48 sequences were generated with ProteinMPNN™, and assembly to the target oligomeric structure validated with AF2 (these evaluations are far fewer in number compared to the thousands of evaluations in the original hallucination trajectories, making overfitting much less likely). We independently evaluated the designs using an updated version of RoseTTAFold™ (RF2) (30) and found that while most of the original AF2 hallucinated sequences were not confidently predicted to fold to the hallucinated structures (see FIG. 9), following ProteinMPNN™ redesign almost all were predicted to fold correctly.

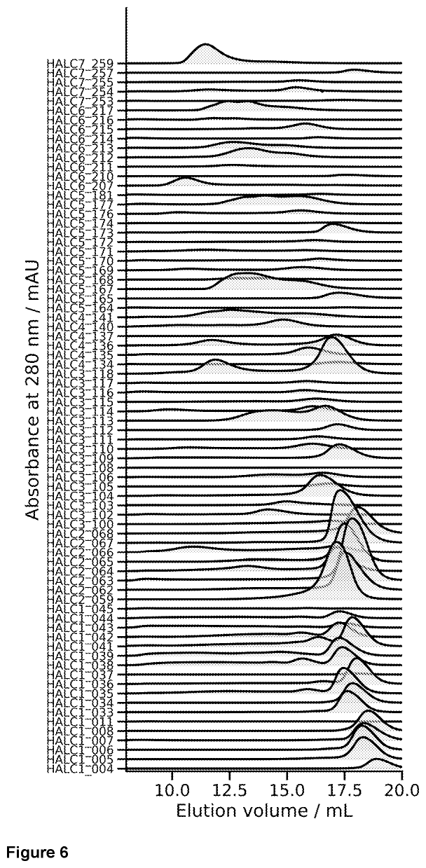

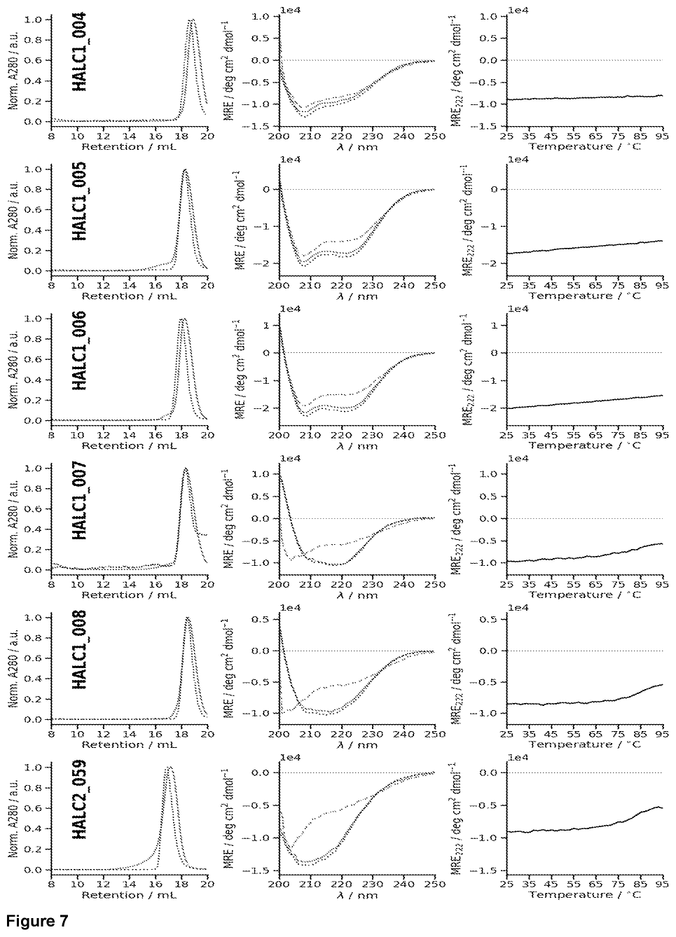

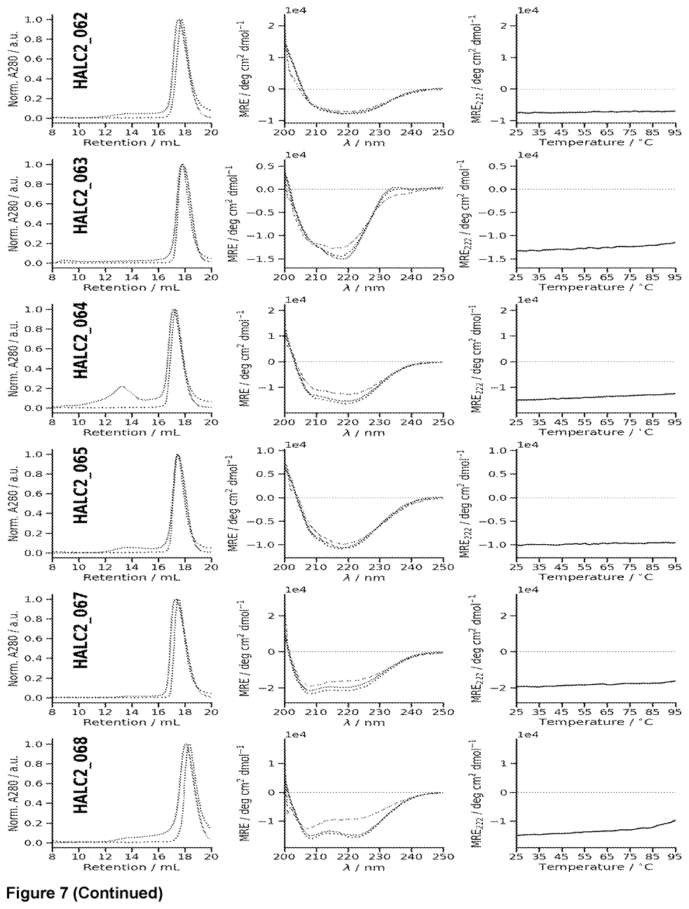

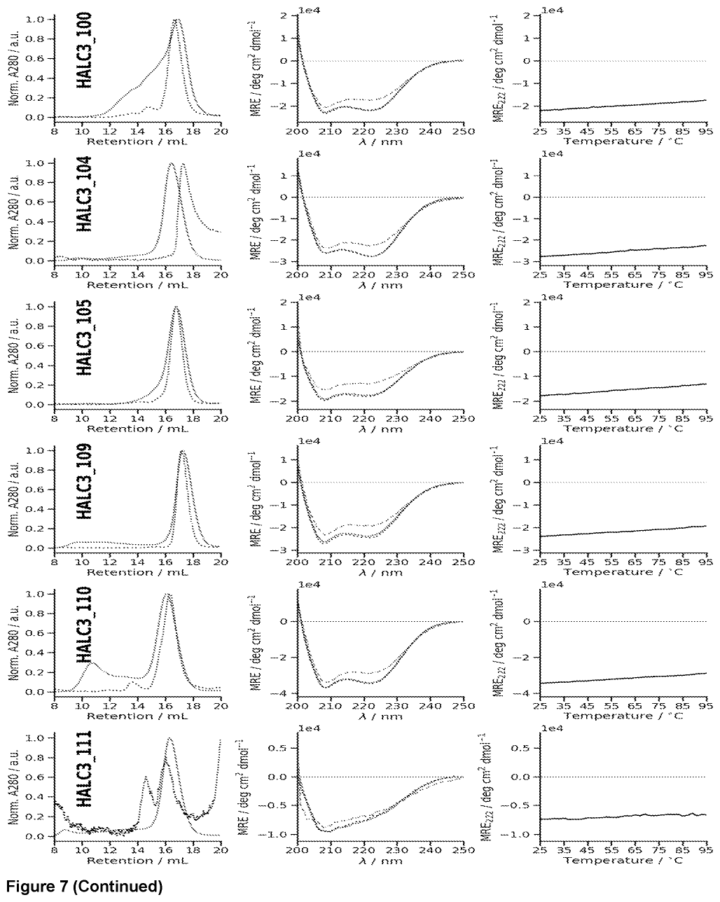

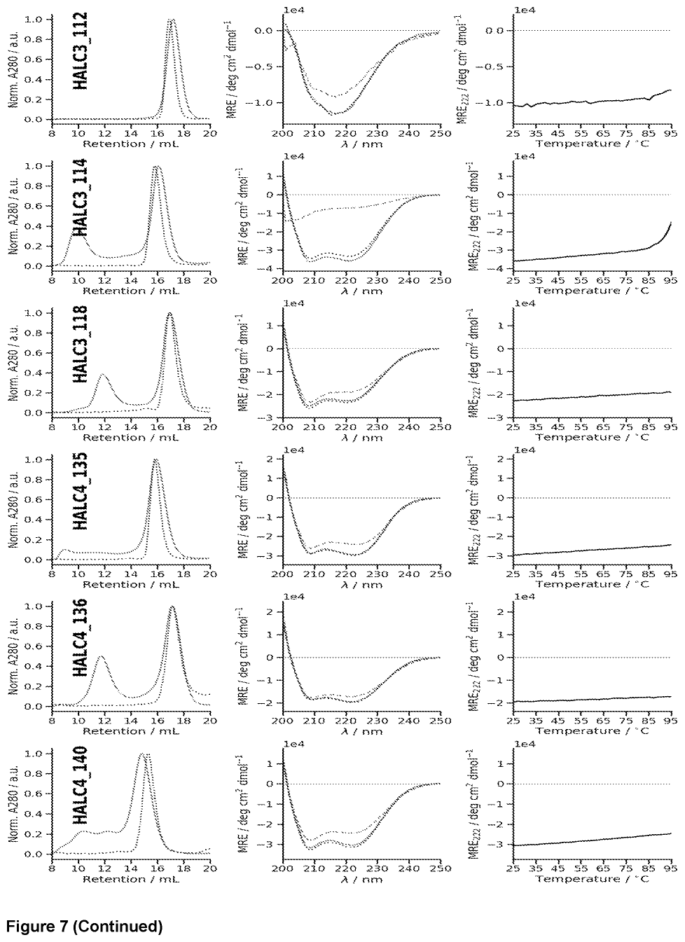

We tested 96 ProteinMPNN™-designed HALs with pLDDT>0.75 and RMSD to original backbone<1.5 Å and found that 71/96 (74%) showed of high levels of soluble expression (median yield: 247 mg per liter of culture-equivalent), 50/96 (52%) had a SEC retention volume consistent with the oligomeric size (of which 30 (60%) were monodisperse) (FIG. 1F and FIG. 6), and at least 21/96 (22%) had the correct oligomeric state when assessed by SEC-Multi Angle Light Scattering (SEC-MALS) (FIG. 1G). Furthermore, CD analysis of the soluble samples indicated that 67/71 (96%) had secondary structure contents consistent with the designs (FIG. 7). These success rates are in stark contrast to those of the original AF2 sequences, indicating that the MCMC hallucination procedure generates viable backbones, but over-fitted sequences, and highlighting the power of ProteinIV1PNNTM to generate sequences which fold to a given backbone structure (FIG. 1E). We assessed the thermal stability of the 71 soluble HALs by CD spectroscopy, and found that 54 maintained their secondary structure up to 95° C. (FIG. 7). SEC characterization of the heated-treated samples indicated that most designs retained their oligomeric state, suggesting that the HAL assemblies are thermostable (FIG. 1H, 7) (Exemplary sequences shown in Table 1).

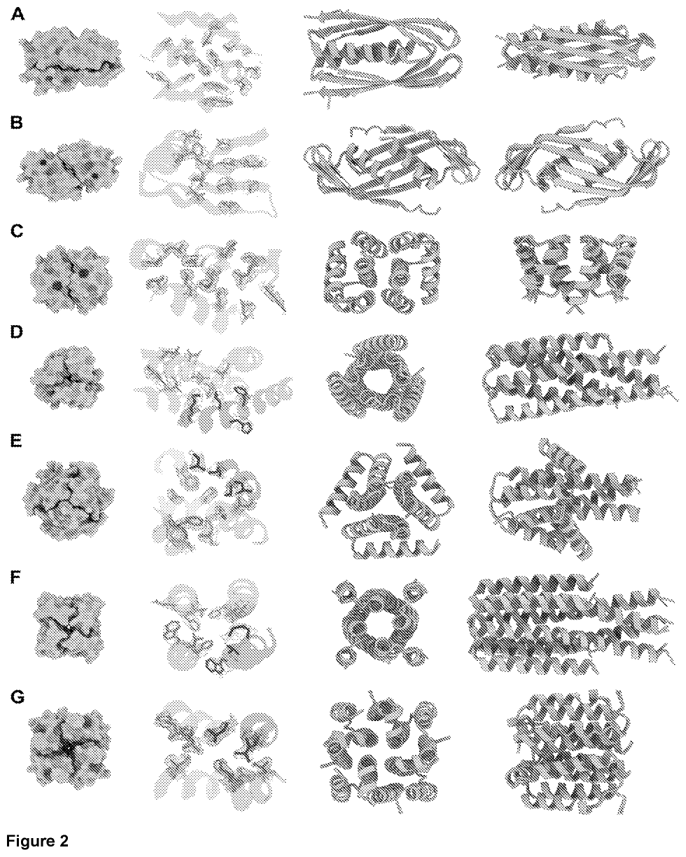

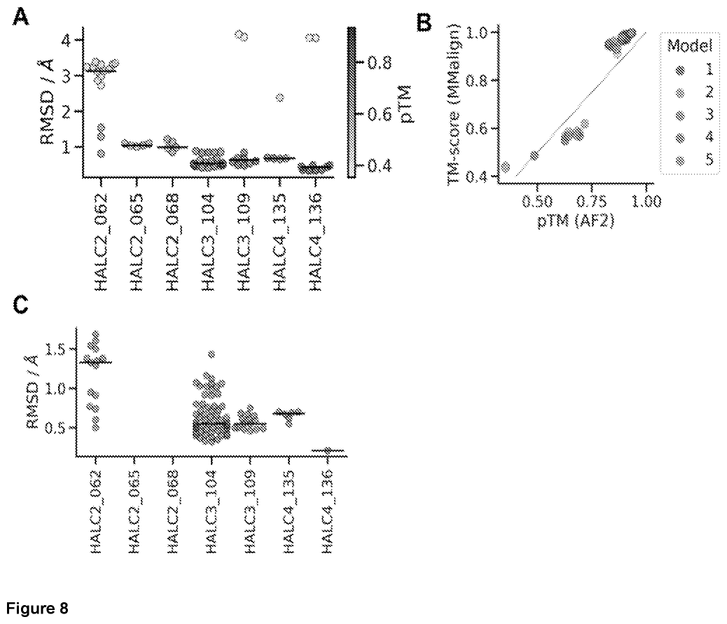

To evaluate design accuracy we attempted crystallization of 19 designs and succeeded in solving crystal structures for seven (three C2s, two C3s and two C4s) (FIG. 2). All crystal structures had the correct oligomerization state and closely matched the design models (median Cα RMSD of 0.6 Å across all designs, FIG. 2 and FIG. 8). The side chain conformations in the crystal structures also closely match those in the design models (FIG. 2).

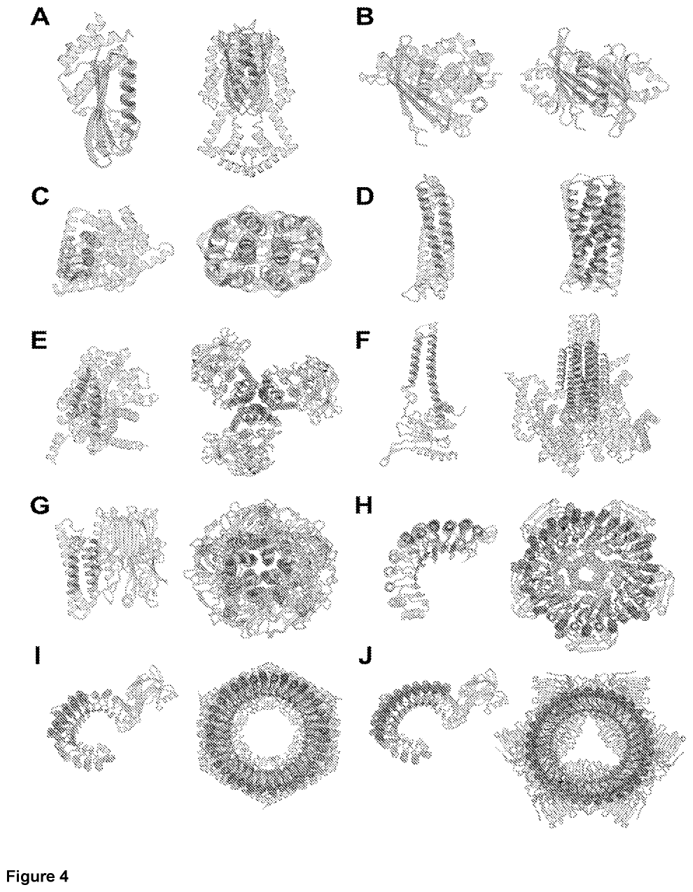

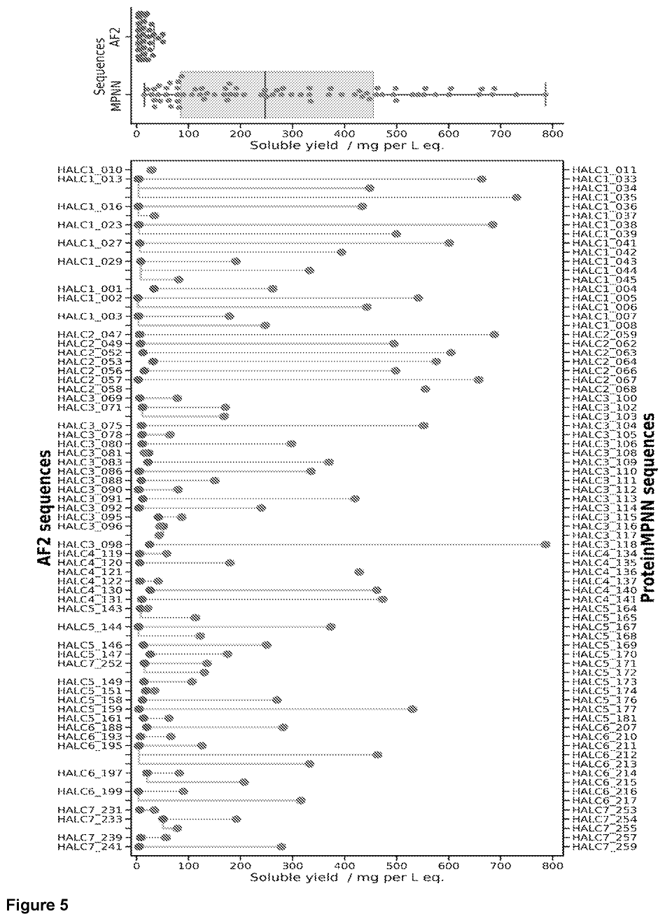

The solved structures exhibit striking diversity with many intricate structural features. HALC2_062 (FIG. 2A) is a three-layer homo-dimer with a single helix from each protomer packed together between two outer (3-sheets (one from each protomer), while HALC2_065 (FIG. 2B) is also a mixed α/β homo-dimer, but has a single, continuous β-sheet shared between both chains, which wraps around two perpendicular paired helices. These two hallucinated structures are very different from anything deposited in the PDB, with TM-scores to their best matches of and 0.54 respectively (FIG. 4A-B, Table 2). HALC2_068 (FIG. 2C) is a fully helical dimer with an extensive interface formed by 6 interacting helices (3 from each protomer), with a single perpendicular helix buttressing the interfacial helices. Despite the absence of secondary structure complexity and long-range contacts, this design also differs significantly from its closest structural relative in the PDB (TM-score: 0.57, FIG. 4C, Table 2). HALC3_104 (FIG. 2D) is a homo-trimeric coiled-coil, with a central bundle of three helices, augmented by an outer-ring of three shorter helices that lay in the groove formed by adjacent protomers. Unsurprisingly given the simplicity of this topology, there is a close structural match in the PDB (TM-score: 0.88, FIG. 4D, Table 2). HALC3_109 (FIG. 2E) is a homo-trimeric three-layer all-helical structure, with three inner helices splaying outwards to contact two additional helices from the same protomers at angles of roughly 25° and 90°; the closest assembly in the PDB has a TM-score of 0.69 (FIG. 4E, Table 2). HALC4_135 (FIG. 2F) is a coiled-coil composed of helical hairpins reminiscent of HALC3_104, but with C4 symmetry instead of C3, and a discontinuous superhelical twist. Despite its simple topology, the closest structural homologue of this design has a TM-score of only 0.59 (FIG. 4F, Table2). HALC4_136 (FIG. 2G) is composed of 3-helix protomers with eight outer helices encasing four almost fully hydrophobic inner helices, where two of the helices are rigidly linked through a 90° helical kink. The closest match in the PDB has a TM-score of 0.71, but the matched structure has C5 symmetry rather than the C4 symmetry of the design and crystal structure.

Next, we sought to generate HALs of increased complexity across longer length-scales by extending the design specifications to structures of higher symmetry (up to C42) and longer assembly sequence length (up to 1800 residues). To generate multiple possible oligomers from a single structure, we specified the MCMC trajectories as single-chains with internal sequence symmetry, with the goal of generating structure-symmetric repeat proteins that could be split into any desired oligomeric assembly compatible with factorization (e.g. C15 into a pentamer, shorthanded as C15-5). To maximize the exploration of the design space while minimizing use of computational resources, we devised an evolution-based computational strategy: many short MCMC trajectories (<50 steps) outputs were clustered by structure prediction confidence metrics (pLDDT and pTM), and then used to seed new trajectories (see Supplementary Materials). Using this approach, we hallucinated cyclic homo-oligomers from C5 to C42 ranging from 7 to 14 nm (median: 10 nm) along their largest dimension, which were then divided into homo-trimers, tetramers, pentamers, hexamers, heptamers, octamers, and dodecamer, and the backbones were re-designed with ProteinMPNN™ (FIG. 1C). While the α/β topology of some of these larger HALs is reminiscent of natural Leucine Rich Repeats (LRRs, (31)), which is reflected by a median highest protomer TM-scores of 0.64, these ring-shaped structures differ considerably from the horseshoe folds of LRRs that do not close into cyclic structures. The closest oligomer structures in the PDB have a median TM-score of 0.47, and BLAST sequence similarity searches for the repetitive sequence motif do not return any significant hits (FIG. 1D); the hallucination process as in the earlier cases clearly generalizes well beyond the training set.

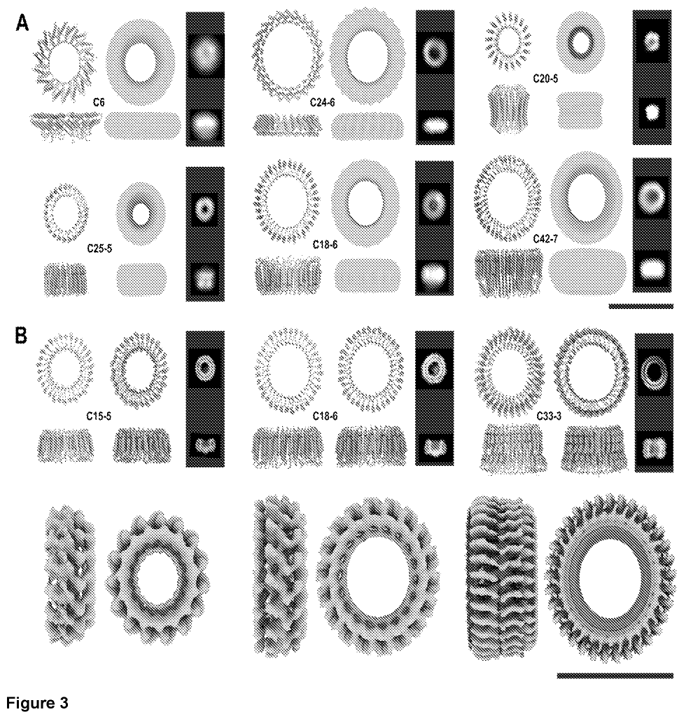

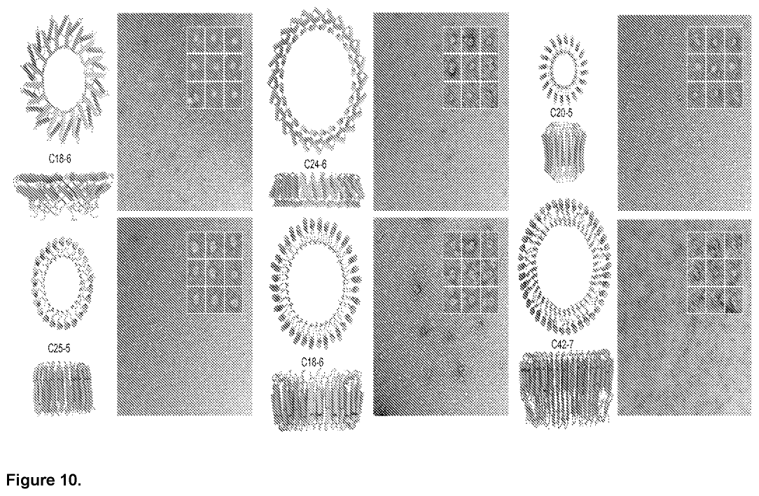

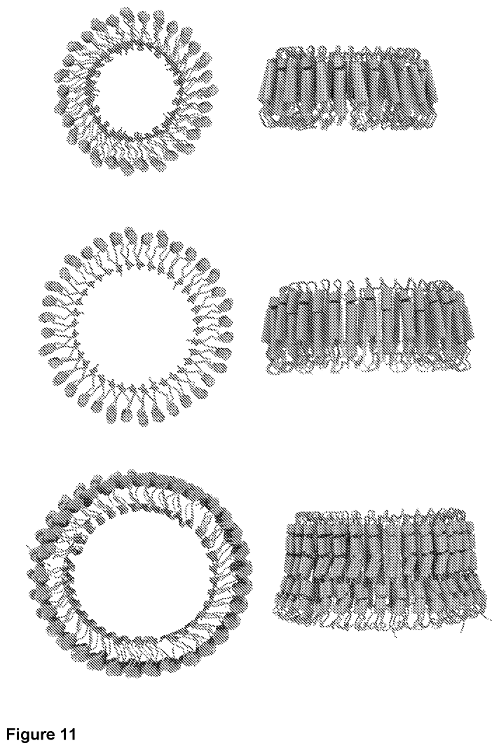

These larger HALs have overall molecular weights greater than 100 kDa, and thus were well-suited for structural characterization by electron microscopy (EM). We subjected soluble large HALs with a SEC retention volume consistent with the size of their oligomeric state to screening by negative stain EM (nsEM). Inspecting the resulting micrographs, we found that all of the designs screened showed monodisperse particles of the expected size and circular shape (FIG. 10). We obtained 2D class averages and 3D ab initio reconstructed electron density maps for six designs (two C5s, three Chs, and one C7) with C6 to C42 internal repeat symmetry that clearly showed low-resolution structural features and diameters consistent with their designs (FIG. 3A, FIG. 11). Next, we selected three designs: one C15 homo-pentamer (HALC5-15_262), one C18 homo-hexamer (HALC6-18_265) and one C33 homo-trimer (HALC3-33_343) for high-resolution single particle cryoEM characterization. We collected datasets that produced 2D class averages with clear secondary structure feature placements, and 3D ab initio reconstruction and refinement yielded 3D electron density maps at 4.38 A, 6.51 A and 6.32 A resolution respectively. HALC5-15 262 was designed as a homo-hexamer, but structure prediction calculations were more consistent with a pentameric structure with a nearly identical protomer internal conformation and a very slightly shifted subunit interface; the cryoEM structure is also a pentamer with an Ca RMSD of 1.69 Å to the predicted structure.

The hallucinated rings are giant structures quite unlike anything in the PDB. The three rings solved by cryoEM, HALC5-15_262, HALC6-18_265 and HALC3-33_343, are 87 Å, 99 Å and 100 Å in diameter and 40 to 50 Å high, with a continuous parallel (3-sheet in the lumen of the pore, and outer helices that enforce the curvature and closure of the ring. HALC3-33_343 has a simple helix-loop-sheet structural motif as the repeating unit, while in HALC5-15_262 and HALC6-18_265, the repeating unit contains two distinct helix-loop-sheet elements, which produces an alternating helical outer pattern clearly observable in the 2D class averages. While both structures have reasonable matches to LRRs for their protomers (TM-score of 0.65 for both, but to different structures), the oligomers are strikingly different from any natural protein, with TM-scores of 0.48 and 0.49 respectively (FIG. 4H-I). HALC3-33_343 has an unusual internal loop region breaking the outer helices midway in the repeat, producing a widening of the ring on one side, which is clearly visible in the cryoEM reconstruction; the protomer has a low TM-score (0.48) despite having an LRR-like topology, and the oligomer is even further from anything currently known (TM-score: 0.41) To our knowledge, these designs are the largest cyclic homo-oligomers designed de novo to date, and the sophistication of the fold, topology, and high sequence and structural symmetry rivals that in nature: the highest cyclic symmetry recorded in the PDB for naturally occurring proteins is C39 (Vault proteins (32), PDB 4HL8 and 7PKY), and there are no closed symmetric a/(3 ring-like structures.

### Conclusion

Our deep learning-based approach to designing cyclic homo-oligomers jointly generates protomers and their oligomeric assemblies without the need for a hierarchical docking approach. We report a rich assortment of de novo protein homo-oligomers across the nanoscopic scale, with broad topological diversity while maintaining design constraints such as symmetry and oligomeric state. These hallucinated oligomers differ substantially from natural oligomers in both sequence (median lowest BLAST™ E-value against UniRef100 of 1.3 for the repeated sequence motifs, FIG. 1D; Table 3)) and structure (median best TM-scores against the PDB for the protomers and oligomers of 0.67 and 0.57 respectively, FIG. 1C); our computational pipeline interpolates and extends native fold-space rather than simply recapitulating memorized protein structures, demonstrating the power of deep learning to explore previously uncharted regions of the design landscape (FIG. 1B). Our results also highlight the power of the ProteinMPNN™ method for protein sequence design: of the 30 out of the 192 designs evaluated experimentally by either SEC-MALS, nsEM, cryoEM, or X-ray crystallography, 27 had the intended oligomeric state, and 7 of 19 for which crystallization was attempted formed diffracting crystals (this is a considerably higher crystallization success rate than typical for Rosetta™ de novo designs, and suggests that ProteinMPNN™ may generate protein surfaces more likely to form crystal contacts).

The high level of abstraction associated with the specification of a loss function enables the design of complex structures with minimal user input, facilitating the design process and making it accessible to non-experts, while generating a rich array of solutions with high experimental success rates. The formalism described here can be extended to other types of complex design tasks, including the design of higher order point group symmetries, arbitrary symmetric or asymmetric hetero-oligomeric assemblies, oligomeric scaffolding of existing functional domains, and design of multiple states, provided a loss function describing the solution can be formalized and computed. Computational requirements and hardware memory limitations become bottlenecks for hallucination of increasingly large structures; the development of computationally less expensive structure prediction methods with fewer parameters, for instance limited to backbone generation, as well as faster-converging algorithms for navigating the sequence space, will further increase the power of the method.

### REFERENCES

1. H. Garcia-Seisdedos, C. Empereur-Mot, N. Elad, E. D. Levy, Proteins evolve on the edge of supramolecular self-assembly. Nature. 548, 244-247 (2017).

2. I. G. Johnston, K. Dingle, S. F. Greenbury, C. Q. Camargo, J. P. K. Doye, S. E. Ahnert, A. A. Louis, Symmetry and simplicity spontaneously emerge from the algorithmic nature of evolution. Proc. Natl. Acad. Sci. 119, e2113883119 (2022).

3. S. E. Ahnert, J. A. Marsh, H. Hernandez, C. V. Robinson, S. A. Teichmann, Principles of assembly reveal a periodic table of protein complexes. Science. 350, aaa2245 (2015).

4. wwPDB consortium, Protein Data Bank: the single global archive for 3D macromolecular structure data. Nucleic Acids Res. 47, D520—D528 (2019).

5. D. S. Goodsell, A. J. Olson, Structural Symmetry and Protein Function. Annu. Rev. Biophys. Biomol. Struct. 29, 105-153 (2000).

6. T. Handel, W. F. DeGrado, De novo design of a Zn2+-binding protein. J. Am. Chem. Soc. 112, 6710-6711 (1990).

7. P. B. Harbury, J. J. Plecs, B. Tidor, T. Alber, P. S. Kim, High-Resolution Protein Design with Backbone Freedom. Science. 282, 1462-1467 (1998).

8. J. A. Fallas, G. Ueda, W. Sheffler, V. Nguyen, D. E. McNamara, B. Sankaran, J. H. Pereira, F. Parmeggiani, T. J. Brunette, D. Cascio, T. R. Yeates, P. Zwart, D. Baker, Computational design of self-assembling cyclic protein homo-oligomers. Nat. Chem. 9, 353-360 (2017).

9. A. R. Thomson, C. W. Wood, A. J. Burton, G. J. Bartlett, R. B. Sessions, R. L. Brady, D. N. Woolfson, Computational design of water-soluble α-helical barrels. Science. 346, 485-488 (2014).

10. P.-S. Huang, K. Feldmeier, F. Parmeggiani, D. A. Fernandez Velasco, B. Hocker, D. Baker, De novo design of a four-fold symmetric TIM-barrel protein with atomic-level accuracy. Nat. Chem. Biol. 12,29-34 (2016).

11. P.-S. Huang, G. Oberdorfer, C. Xu, X. Y. Pei, B. L. Nannenga, J. M. Rogers, F. DiMaio, T. Gonen, B. Luisi, D. Baker, High thermodynamic stability of parametrically designed helical bundles. Science. 346, 481-485 (2014).

12. S. E. Boyken, Z. Chen, B. Groves, R. A. Langan, G. Oberdorfer, A. Ford, J. M. Gilmore, C. Xu, F. DiMaio, J. H. Pereira, B. Sankaran, G. Seelig, P. H. Zwart, D. Baker, De novo design of protein homo-oligomers with modular hydrogen-bond network-mediated specificity. Science. 352, 680-687 (2016).

13. J. B. Bale, S. Gonen, Y. Liu, W. Sheffler, D. Ellis, C. Thomas, D. Cascio, T. 0. Yeates, T. Gonen, N. P. King, D. Baker, Accurate design of megadalton-scale two-component icosahedral protein complexes. Science. 353, 389-394 (2016).

14. I. Vulovic, et al., Generation of ordered protein assemblies using rigid three-body fusion. Proc. Natl. Acad. Sci. 118, e2015037118 (2021).

15. Y. Hsia, R. Mout, W. Sheffler, N. I. Edman, I. Vulovic, Y.-J. Park, R. L. Redler, M. J. Bick, A. K. Bera, A. Courbet, A. Kang, T. J. Brunette, U. Nattermann, E. Tsai, A. Saleem, C. M. Chow, D. Ekiert, G. Bhabha, D. Veesler, D. Baker, Design of multi-scale protein complexes by hierarchical building block fusion. Nat. Commun. 12, 2294 (2021).

16. C. E. Correnti, J. P. Hallinan, L. A. Doyle, R. O. Ruff, C. A. Jaeger-Ruckstuhl, Y. Xu, B. W. Shen, A. Qu, C. Polkinghorn, D. J. Friend, A. D. Bandaranayake, S. R. Riddell, B. K. Kaiser, B. L. Stoddard, P. Bradley, Engineering and functionalization of large circular tandem repeat protein nanoparticles. Nat. Struct. Mol. Biol. 27, 342-350 (2020).

17. D. D. Sahtoe, F. Praetorius, A. Courbet, Y. Hsia, B. I. M. Wicky, N. I. Edman, L. M. Miller, B. J. R. Timmermans, J. Decarreau, H. M. Morris, A. Kang, A. K. Bera, D. Baker, Reconfigurable asymmetric protein assemblies through implicit negative design. Science. 375, eabj7662 (2022).

18. I. Anishchenko, S. J. Pellock, T. M. Chidyausiku, T. A. Ramelot, S. Ovchinnikov, J. Hao, K. Bafna, C. Norn, A. Kang, A. K. Bera, F. DiMaio, L. Carter, C. M. Chow, G. T. Montelione, D. Baker, De novo protein design by deep network hallucination. Nature. 600, 547-552 (2021).

19. M. Jendrusch, J. O. Korbel, S. K. Sadiq, AlphaDesign: A de novo protein design framework based on AlphaFold (2021), p. 2021.10.11.463937,doi:10.1101/2021.10.11.463937.

20. L. Moffat, J. G. Greener, D. T. Jones, Using AlphaFold for Rapid and Accurate Fixed Backbone Protein Design (2021), p. 2021.08.24.457549, doi:10.1101/2021.08.24.457549.

21. J. Wang, et al, Deep learning methods for designing proteins scaffolding functional sites (2021), p. 2021.11.10.468128, doi:10.1101/2021.11.10.468128.

22. S. Ovchinnikov, P.-S. Huang, Structure-based protein design with deep learning. Curr. Opin. Chem. Biol. 65, 136-144 (2021).

23. C. Norn, et al., Protein sequence design by conformational landscape optimization. Proc. Natl. Acad. Sci. 118, e2017228118 (2021).

24. N. Anand, R. Eguchi, I. I. Mathews, C. P. Perez, A. Derry, R. B. Altman, P.-S. Huang, Protein sequence design with a learned potential. Nat. Commun. 13, 746 (2022).

25. J. Jumper, R. Evans, A. Pritzel, T. Green, M. Figurnov, O. Ronneberger, K. Tunyasuvunakool, R. Bates, A. Z̆idek, A. Potapenko, A. Bridgland, C. Meyer, S. A. A. Kohl, A. J. Ballard, et al., Highly accurate protein structure prediction with AlphaFold. Nature. 596, 583-589 (2021).

26. J. Xu, Y. Zhang, How significant is a protein structure similarity with TM-score=0.5 Bioinformatics. 26, 889-895 (2010).

27. Inceptionism: Going Deeper into Neural Networks. Google AI Blog, (ai.googleblog.com/2015/06/inceptionism-going-deeper-into-neural.).

28. A. Nguyen, J. Yosinski, J. Clune, Deep Neural Networks are Easily Fooled: High Confidence Predictions for Unrecognizable Images (2015), (arxiv.org/abs/1412.1897).

29. K. Simonyan, A. Vedaldi, A. Zisserman, Deep Inside Convolutional Networks: Visualising Image Classification Models and Saliency Maps (2014), (arxiv.org/abs/1312.6034).

30. M. Baek, et al., Accurate prediction of protein structures and interactions using a three-track neural network. Science. 373, 871-876 (2021).

31. B. Kobe, J. Deisenhofer, The leucine-rich repeat: a versatile binding motif. Trends Biochem. Sci. 19, 415-421 (1994).

32. P. Guerra, M. Gonzalez-Alamos, A. Llauro, A. Casafias, J. Querol-Audi, P. J. de Pablo, N. Verdaguer, Symmetry disruption commits vault particles to disassembly. Sci. Adv. 8, eabj7795 (2022).

33. A. Courbet, et al., Computational design of mechanically coupled axle-rotor protein assemblies. Science. 376, 383-390 (2022).

### Materials and Methods

### Computational Design Strategy

We reasoned that the ability of AF2 to predict oligomers could be employed to design such structures using a MCMC search in sequence space in combination with a suitable loss function. The advantage of such a method is its ability to jointly optimize the protomer and oligomer structures, without putting any constraints on the nature of the protomer itself (e.g. the requirement to adopt a well-folded structure in isolation as is typically the case for docking approaches). We employed simplifications during AF2 predictions to reduce computational cost, and defined a composite loss function composed of structure quality terms and a geometric term.

MCMC trajectories were initialized with a random protomer sequence of specified length, with the composition of amino acids respecting the BLOSUM62 background frequencies. Cysteines were disallowed for all hallucinations. Protomers sequences were concatenated to generate oligomeric assemblies during AF2 prediction: chain breaks in the concatenated protomer sequences were specified by re-indexing residues after the break with a 200 increment, resulting in AF2 predicting them as separate chains. To reduce computational costs the number of recycles was set to 1, the number of ensembles was also set to 1, and AMBER relax was not performed. After each prediction losses were computed on the AF2 prediction confidence metrics (pLDDT, pTM, pAE) as well as the coordinates of the predicted structure.

Mean AF2 pLDDT and AF2 pTM scale between 0 and 1, where higher values are better, thus the loss (by definition the objective to minimize) was calculated for each as one minus their respective values. For enforcing cyclic symmetry we computed a cyclic loss term defined as the standard deviation between the center of mass of adjacent protomers (computed on Ca). Minimizing this value enforces cyclic symmetry.

The loss functions computed to generate all cyclic oligomers <=C7 was:

Dual_cyclic: loss=1−0.5*(AF2pTM+AF2pLDDT)+standard deviation(center of masses)

After an initial prediction, mutations were introduced in the protomer sequences (tied positions), and the structure re-predicted. Positions with low pLDDT values (lowest half) were targeted, and mutations were chosen based on the BLOSUM62 substitution frequencies. The number of mutations at each step was linearly decayed over the course of the trajectory starting from 3 per protomer down to 1.

Simulated annealing was employed during optimization, with the starting temperature set to 0.01 and the half-life of the exponential decay set to 500 steps. Mutations were accepted or rejected according to the Metropolis criterion

Modest computational means were sufficient to hallucinate assemblies up to C7 with protomer lengths of 65 amino acids. The largest C7 assemblies required a week on a single CPU with 6 GB of memory to generate 300 steps, which can be sufficient for convergence (pLDDT>0.70 and pTM>0.70) . For smaller assemblies (e.g. a C3 with protomers composed of 65 amino acids) approximately 500 steps per day could be obtained on a single CPU with 5 GB of memory.

The structures generated from AF2 hallucination were sequence re-designed with ProteinMPNN™ using only the restrictions that protomer sequences in the oligomeric assembly were tied to be identical, and cysteines were disallowed. For each backbone 24-48 sequences were generated with ProteinMPNN™ using a temperature of 0.2. The quality of these sequences was assessed with AF2 using all 5 models (model 1-5ptm), checking both the confidence metrics and the structural recapitulation of the original backbone geometry. Sequences were filtered on having AF2 pLDDT>0.75, and a RMSD to the original protomer backbone <1.5 Å (computed with TMalign, (34, 35)). For each original backbone the four designs with highest AF2 pLDDT were inspected by eye, and up to three MPNN sequences per original input backbone were ordered for experimental testing.

### RoseTTAFold™ Prediction of Oligomers

An updated version of RoseTTAFold TM was used to evaluate designed oligomers. This RoseTTAFold™ model has multiple architectural improvements over the original published model, including; 1) use of a 3D track from the beginning, with coordinates from a template or the previous recycling round, 2) communication between 1D, 2D, and 3D tracks through attention biasing, and 3) use of recycling that executes the network multiple times with the updated input embeddings based on outputs from the previous cycle. The model was trained with 3 recycling steps. The training dataset comprised; 1) both single-chain and biologically relevant complex structures from the PDB released before Apr. 30, 2020, and 2) AlphaFold2™ model structures for UniRef50 representatives. For the examples used during training that were oligomers, we added 200 to the residue numbers of the following subunits to indicate chain breaks to the network. Two rounds of model training were performed; 1) an initial training (200 epochs, with 25600 examples per epoch and a batch size of 64) based on the masked language recovery loss, distogram prediction loss, predicted LDDT loss, and FAPE loss followed by, 2) fine-tuning (50 epochs, with 25600 examples per epoch and a batch size of 64) with additional loss terms on bond geometry and van der Waals scoring function. We trained the model with a crop size of 256 residues, and then fine-tuned it with a larger crop (384 residues). The AdamW

Optimizer with default pytorch parameters was used. For the initial training we linearly increased the learning rate to 0.001 over the first 1000 optimization steps, and further decreased the learning rate by a factor of 0.95 for every additional 5000 optimization steps. The fine-tuning stage started from the pre-trained model weights, and used the lower learning rate (0.0005), no warm-up steps, and the same step-wise learning rate decay.

During inference, we added 200 to the residue indices of subsequent subunits to indicate chain breaks, as we did during model training. The model was recycled 20 times, and the predicted structure having the highest LDDT estimation was selected. The oligomer structure predictions were generated from the designed sequence only, without any MSA or template information.

### Comparison to Natural Proteins

The outputs generated during AF2 hallucination and ProteinMPNN™ re-design were assessed for their sequence and structure novelty. Sequence homologues were searched using BLAST (Protein-Protein BLAST version 2.11.0+) against UniRef100 (snapshot from Mar. 2, 2022; Table 3) and the E-value of the best hit reported. Both the sequence of the protomer as well as the repeated sequence motif were queried. In the case of small HALs, the protomer and repeated sequence motif were equivalent, but not in the case of large HALs (i.e. HALCX-Y), where protomers are composed of multiple repeated sequence motifs. Structural comparisons to published structures were performed at the protomer level (using TMalign version 20190425) against the PDB (snapshot from Apr. 15, 2022) and over the whole oligomer (using MMalign version 20210816) against all biounits assigned in the PDB (snapshot from Apr. 15, 2022). In both cases results are reported as TM-score.

### Representation of the Structural Space

A representation of the structural space covered by the outputs of the hallucination trajectories compared to all de novo cyclic structures deposited in the PDB is shown in FIG. 1B. The plot was obtained by Multidimensional scaling (as implemented in the sklearn python library) on a pre-computed pairwise distance matrix. Pairwise distances were defined as 1-TM-score, and the score computed with TMalign™ (version 20190425). The list of 162 de novo cyclic structures was obtained by using the following gate on a snapshot of the PDB from Apr. 17, 2022:

Entry Polymer Composition==homomeric protein & Polymer Entity Sequence Length >=40 & Structure Keywords contains ‘de novo’ & Type==Cyclic

lec5,1g6u, 1jm0, 1jmb, 11t1, 1mft, 1ovr, 1ovu, 1ovv, 1u7j, 1u7m, 1uw1, 1vjg, 1y47, ly66, 2gjf, 2gjh, 2i7u, 2jst, 2kik, 2mg4, 2p05, 2p09, 2wqh, 2zgd, 2zgg, 3cwo, 3dgo, 3lt8, 3lt9, 3lta, 3ltb, 3ltc, 3ltd, 3m22, 3m24, 3mlg, 3o10, 3rhu, 3tdm, 3tdn, 3v1b, 3v1c, 3v1d, 3v1e, 3v1f, 3vjf, 3ww7, 3ww8, 3wwb, 3wwf, 4db8, 4dba, 4etj, 4f2v, 4glu, 4hxt, 4loa,4lpu, 4lpv, 4lpw, 4lpx, 4lpy, 4m6a, 4ndj, 4ndk, 4ney, 4nez, 4o60, 4ow4, 4pww, 4qfv, 4rjv, 4wpy, 4yfo, 4yxy, 4zcn, 4zxz, 5a0o, 5bvb, 5c39, 5di5, 5dn0, 5dns, 5j0j, 5j0k, 5j01, 5j10, 5j21, 5j73, 5k7v, 5kay, 5kba, 5kwd, 510p, 5od9, 5tph, 5u35, 5vl4, 5ys7, 6ff6, 6g6q, 6idc, 6iei, 6kos, 6m6z, 6msq, 6msr, 6m9h, 6naf, 6nek, 6nla, 6nx2, 6ny8, 6nye, 6nyi, 6nyk, 6nz1, 6nz3, 6o0c, 6o0i, 6o35, 6gsh, 6tjb, 6tjc, 6tjd, 6uls, 6v8e, 6veh, 6w40, 6w6x, 6wxo, 6wxp, 6xh5, 6xi6, 6xns, 6xr2, 6xss, 6xt4, 6y7n, 6zv9,7ax0,7bww,7dns,7k3h,7kxs,7m0q,7nbi

### Plasmid Construction

Plasmids for expressing HALs were constructed from synthetic DNA according to the following procedure: Linear DNA fragments (Integrated DNA Technologies, IDT eblocks) encoding design sequences and including overhangs suitable for a Bsal restriction digest were cloned into custom target vectors using Golden Gate Assembly. All subcloning reactions resulted in C-terminally HIS-tagged constructs.

The entry vectors for Golden Gate cloning are modified pET29b+vectors that contain a lethal ccdb gene between the Bsal restriction sites that is both under control of a constitutive promoter and in the T7 reading frame. The lethal gene reduces background by ensuring that plasmids that do not contain an insert (and therefore still carry the lethal gene) kill transformants. The vectors were propagated in ccdb resistant NEB Stable cells (New England biolabs C3040H, always grown from fresh transformants). Plasmids were deposited with Addgene.

Golden Gate reactions (5 uL per well) were set up on a 96 well PCR plate as:

| | | | | |
|---|---|---|---|---|
| 10 × T4 Buffer | | 0.5 | uL | 10 × T4 Buffer (New England Biolabs B0202S) |
| Vector | | 10-20 | fmol | Vector (either LM627 or LM670) |
| BsaI-HFv2 | 3 U | 0.15 | uL | BsaI-HFv2 (New England Biolabs R3733L) |
| T4 Ligase | 100 U | 0.25 | uL | T4 Ligase (New England Biolabs M0202L) |

+ (20-40 fmol) linear DNA fragment, typically 1 uL of 10 ng/uL stock

Complete with nuclease-free water to 5 uL total reaction volume.

The reactions were incubated at 37° C. for 20 minutes, followed by 5 min at 60° C. in a thermocycler (Biorad T100) with the lid heated to 105° C.

### Small-Scale Protein Solubility Screen

For initial solubility screens, Golden Gate reaction mixtures were transformed into BL21(DE3) (New England Biolabs) as follows: 1 uL of reaction mixture was added to 6-8 uL of competent cells on ice in a 96 well PCR plate. The mixture was incubated on ice for 30 minutes, then heat-shocked for 10 s at 42° C. in a block heater (IKA Dry Block Heater 3), then rested on ice for 2 minutes. Subsequently, 100 uL of room temperature SOC media (New England Biolabs) was added to the cells, followed by incubation at 37° C. with shaking at 1000 rpm on a Heidolph Titramax™ 1000/Incubator 1000.

The transformations were then grown in a 96 well deep-well plate (2 mL total well volume) in autoclaved LB media supplemented with 50 μg mL⁻¹ Kanamycin at 37° C. and 1000 rpm. In the following protocols all growth plates were covered with breathable film (Breathe Easier, Diversified Biotech) during incubation.

The following day, glycerol stocks were made from the overnight cultures (100 uL of 50% [v/v] Glycerol in water mixed with 100 uL bacterial culture, frozen and kept at −80° C. Subsequently, two 96 deep well plates were prepared with 900 uL per well of autoclaved Terrific™ Broth II (MP biomedicals) supplemented with 50 μg mL⁻¹ Kanamycin, and 100 uL of the overnight culture were added and grown for 1.5 h at 37° C., 1200 rpm (Heidolph Titramax™ 1000/Incubator 1000). The cultures were then induced with IPTG by adding 10 uL of 100 mM (final concentration approximately 1 mM) per well with an electric repeater pipette (Eppendorf, E4x series), and grown for another 4 h at 37° C., 1200 rpm. Cultures were combined into a single 96 well plate for a total culture volume of 2 mL and harvested by centrifugation at 4000 ×g for 5 min. Growth media was discarded by rapidly inverting the plate, and harvested cell pellets were either processed directly, or frozen at −80° C.

Proteins were purified by HIS tag-based Immobilized metal affinity chromatography (IMAC). Bacterial pellets were resuspended and lysed in 300 uL B-PER chemical lysis buffer (Thermo Fisher Scientific) supplemented with 0.1 mg mL⁻1 Lysozyme (from a 100 mg mL⁻¹ stock in 50% [v/v] Glycerol, kept at −20° C., Millipore Sigma), 50 Units of Benzonase per mL (Merck/Millipore Sigma, stored at −20° C.), and 1 mM PMSF (Roche Diagnostics, from a 100 mM stock kept in Propan-2-ol, stored at room temperature). The plate was sealed with an aluminum foil cover and vortexed for several minutes until the bacterial pellet was completely resuspended (on a Vortex Genie™ II, Scientific Industries). The lysate was incubated, shaking for 5 minutes, before being spun down at 4000×g for 15 minutes. In the meantime, 75 uL of Nickel-NTA resin bed volume (Thermo Scientific, resin was regenerated before each run and stored in 20% [v/v] Ethanol) was added to each well of a 96 well fritted plate (25 μm frit, Agilent 200953-100). To increase wash step speed, the resin was equilibrated on a plate vacuum manifold (Supelco™, Sigma) by drawing 3×400 uL of Wash buffer (20 mM Tris, 300 mM NaCl, 25 mM Imidazole, pH 8.0) over the resin using the vacuum manifold at its lowest pressure setting.

The supernatant (280 uL) of the lysate was extracted after the spin down and applied to the equilibrated resin and allowed to slowly drip through over ˜5 minutes. Subsequently the resin was washed on the vacuum manifold with 3×400 uL of Wash buffer. Lastly the fritted plate spouts were blotted on paper towels to drain excess Wash buffer. Then 250 uL of Elution buffer (20 mM Tris, 300 mM NaCl, 500 mM Imidazole, pH 8.0) was applied to each well and incubated for 5 minutes before eluting the protein by centrifugation at 1500×g for 5 minutes into a 96 well collection plate. Eluate was stored at 4° C.

Screening samples for EM and initial SDS-PAGE (Biorad Criterion™ 26-well stain free-anykD) analysis to assess solubility were prepared using this method. Correct protomer masses were verified by Liquid chromatography-mass spectrometry (LC-MS, Agilent) on soluble eluates. To identify the molecular mass of each protein, intact mass spectra was obtained via reverse-phase LC/MS with an Agilent G6230B TOF on an AdvanceBio™ RP-Desalting column (A: H2O with 0.1% Formic Acid, B: Acetonitrile with 0.1% Formic Acid), and subsequently deconvoluted with Bioconfirm™ using a total entropy algorithm.

### Larger-Scale Protein Expression and Purification For Biophysical Studies

Overnight autoinduction cultures were seeded from the glycerol stocks made for the small scale screen. Growth media was TB-II autoinduction media: TB-II (Terrific Broth™ II, MP biomedicals-prepared according to manufacturer's specifications: 50 g/L, autoclaved) supplemented with Studier 5052 components from a 50× stock (final concentrations: 5 g/L glycerol, 0.5 g/L dextrose, 2 g/L lactose monohydrate), and 2 mM MgSO₄.

For the initial screen of 150 AF2 hallucinations, 50 mL cultures were grown in 250 mL baffled flasks (24 h, 37° C., 250 rpm). For the subsequent screen of the MPNN designed sequences, 15 mL cultures were grown in 125 mL baffled flasks (16 h, 37° C., 250 rpm). Cultures were harvested by centrifugation at 4000×g for 5 minutes, and pellets were stored frozen at −80° C., or processed directly.

The parameters for the purification of the initial 150 AF2 based hallucinations and the MPNN redesigned sequences are given as (AF2|MNN) differed slightly because of differences in expression culture volume (50 mL |15 mL)

For protein purification, pellets were resuspended in (10 mL |5 mL) Wash buffer (20 mM Tris, 300 mM NaCl, 25 mM Imidazole, pH 8.0 at room temperature, supplemented with 0.1 mg mL⁻¹ Lysozyme, 0.01 mg mL⁻¹, Deoxyribonuclease I (DNAse I, Millipore Sigma), 1 mM PMSF) by vortexing for several minutes until the pellet was fully resuspended. The resuspension was sonicated (Qsonica, Q500 with a: 4 pronged horn |24 pronged horn) as 10 s ON, 10 s OFF, (45% |80%) amplitude for 5 minutes of total ON time, and samples were kept on ice during the whole procedure.

The sonicated lysate was centrifuged at (14000×g |14000×g) for 15-45 minutes to remove the insoluble fraction. Plates with 25 μm bottom frits with (24 |48) wells (Agilent 201415-100 |201003-100) were filled with (1 mL |0.5 mL) of bed Ni-NTA resin (Qiagen or Thermo Fisher), and equilibrated with three rinses of Wash buffer (at least 30 resin bed volumes) on a vacuum manifold as described above.

The fritted plate spouts were closed with parafilm, and the supernatant was added to each well. The plate was sealed and incubated lightly agitated for 30 minutes. The supernatant was drained from the resin, and the resin bed washed three times with (10 mL |5 mL) of Wash buffer (at least 30 resin bed volumes) on the vacuum manifold. Excess Wash buffer was blotted from the spouts on paper towels, and the resin was pre-eluted with 80% resin bed volume of Elution buffer, followed by protein elution into (1.1 mL |0.8 mL) of Elution buffer (20 mM Tris, 300 mM NaCl, 500 mM Imidazole, pH 8.0).

### Size Exclusion Chromatography (SEC)

IMAC eluates were sterile-filtered through a 96 well filter plate (0.2 μm polyethersulphone (PES) membrane, Agilent 204510-100) by centrifugation at 2000×g for 5 minutes.

Size exclusion chromatography was performed using an autosampler-equipped Akta pure system (Cytiva) on a Superdex™ S200 Increase 10/300 GL column at room temperature. The running buffer was 20 mM Na-PO4, 100 mM NaCl, pH 7.4 at room temperature. Selected fractions (shown in FIG. 7) were pooled and concentrated using Spin filters (3 kDa molecular weight cutoff, Amicon, Millipore Sigma) and stored at 4° C. before downstream characterizations. Protein identities were confirmed by reverse-phase LC-MS as described above.

SEC retention volume to molecular weight equivalencies were calibrated with protein standards (Cytiva LMW and HMW kits for the S75 and S200 columns, respectively).

Samples for electron Microscopy were purified by SEC using a Superdex™ 6 10/300 GL increase column (Cytiva) and TBS running buffer (25 mM Tris pH 8.0, 100 mM NaCl). SEC elution fractions corresponding to the design's theoretical elution volumes were concentrated in TBS prior to structural and biochemical analysis.

### Size Exclusion Chromatography-Multi Angle Light Scattering (SEC-MALS)

Pooled SEC samples were analyzed by SEC-MALS in 20 mM Na-PO4, 100 mM NaCl, pH 7.4 on a Superdex™ 75 10/300 or Superdex™ 200 10/300 column in line with a Heleos multi-angle static light scattering and an Optilab T-rEX™ detector (Wyatt Technology Corporation). Data was analyzed using ASTRA™ (Wyatt Technologies) to calculate the weighted average molar mass (Mw) of the selected species and the number average molar mass (Mn) to determine monodispersity by polydispersity index (PDI)=Mw/Mn.

### Circular Dichroism (CD)

Circular Dichroism was performed on a Jasco 1500 CD spectrometer with a 6 sample rotating turret. Samples were placed in 1 mm pathlength cuvettes (Hellma QS Quartz cell) at concentrations of 0.25 mg mL⁻¹ in 20 mM Na-PO4, 100 mM NaCl, pH 7.4 buffer. The temperature was ramped from 25° C. to 95° C., recording full CD spectra between 200 and 260 nm in 10° C. intervals, and reading at 222 nm in 2° C. intervals. After reaching 95° C. the samples were allowed to cool back to 25° C. before recording a final spectrum. Samples were recovered, filtered over a 0.2 μm PES membrane, and re-run over SEC as described above.

### Crystallography Sample Preparation and Data Collection

19 designs were chosen to undergo crystallization screens. Each design was expressed as described above in 0.5 L cultures. Following affinity purification, each design underwent SEC into SNAC cleavage buffer (100 mM CBES, 100 mM NaCl, 100 mM acetone oxime, 500 mM guanidine HCl, pH 8.6). Following SEC, 2 mM of NiCl₂ was added and the solution was incubated overnight at 37° C. Following cleavage, the solutions containing the cleaved protein products were incubated with 1 mL Ni-NTA resin to bind any uncleaved product, and the flow through was collected. Following SEC into Crystallization buffer (20 mM Tris, 50 mM NaCl, pH 8.0), each sample was concentrated to approximately 15 mg mL⁻¹. The following sitting drop broad screens were set up at room temperature with three protein:crystallization condition ratios (1:1, 1:2, 2:1) using the mosquito pipetting instrument (sptlabtech): Midas™ (Molecular Dimensions), Proplex™ (Molecular Dimensions), JCSG+™ (Molecular Dimensions), Morpheus™ (Molecular Dimensions), Pact Premier™ (Molecular Dimensions), LMB™ (Molecular Dimensions), Index™ (Hampton Research) and PGA™ (Molecular Dimensions). Each was monitored weekly for crystal growth using the JANSi UVEX imaging system.

The following conditions yielded diffracting crystals for our designs: 0.05 M Cesium chloride, 0.1 M MES pH 6.5, 30% Jeffamine™ M-600 (HALC3_104); Morpheus™ condition H5 (HALC3_109); 0.1 M BIS-TRIS pH 6.5, 2.0 M Ammonium sulfate (HALC2_062); 0.2 M Lithium sulfate monohydrate, 0.1 M BIS-TRIS pH 6.5, 25% w/v Polyethylene glycol 3,350 (HALC4_135); 0.1M SPG buffer pH 5 25% w/v PEG 1500 (HALC4_136), 0.04 M Potassium phosphate, 16% PEG 8000, 20% Glycerol (HALC2_068); and 0.2 M Ammonium nitrate pH 6.3, 20% PEG 3350 (HALC2_065). Where required, crystals were cryoprotected with 20% glycerol or 25% ethylene glycol prior to flash freezing in liquid nitrogen. Data collection was done using the Advanced Photon Source synchrotron. Images were integrated using XDS 20220110 (37). Aimless (38) was used for scaling and merging. Phaser™ 2.8 (39) was used for molecular replacement using the design models as search models (either monomer or oligomeric complex). Models were built using Coot 0.9.8 (40) and refined with Phenix ™ refine from Phenix™ 1.20 (41) and RefMac™ (42) from CCP4 7.1 (38) suite. All structures were validated using MolProbity™ 4.5.1(43). Crystallographic statistics are available in Table 4.

Negative Stain Electron Microscopy (nsEM):

SEC fractions corresponding to the designs were concentrated in TBS prior to negative stain EM screening. Samples were then immediately diluted 5 to 150 times in TBS buffer (25 mM Tris, 100 mM NaCl, pH 8.0) depending on the concentration of the samples. A final volume of 5 μL was applied on negatively glow discharged, carbon-coated 400-mesh copper grids (01844-F, TedPella Inc.), then washed with Milli-Q Water and stained using 0.75% uranyl formate as previously described (44). Air-dried grids were then imaged on either a FEI Talos™ L120C TEM (FEI Thermo Scientific) equipped with a 4K×4K Gatan OneView™ camera at a magnification of 57,000× and pixel size of 2.5 Å. Micrographs collection was automated using EPU™ software (FEI Thermo Scientific) and were imported into CisTEM™ software (45) or cryoSPARC™ software (46, 47). CTF estimation was done with CTFFIND4 and a circular blob picker was used to select particles which were then subjected to 2D classification. Ab initio reconstruction and homogeneous refinement in Cn symmetry were used to generate 3D electron density maps. All EM maps can be found in supplementary data.

### CryoEM Sample Preparation and Data Collection:

CryoEM grids were prepared by diluting protein samples with TBS 1 to 10 times immediately before applying 3.5 μL to glow-discharged 400 mesh, C-flat, 2 micron holes, 2 micron spacing, CF-2/2-4C (CF-224C-100) (Electron Microscopy Sciences) cryoEM grids. For some samples, multiple blots were applied in order to obtain the best particle density. All grids were blotted using a blot force of 0 and 5.5 second blot time at 100% humidity and 4° C. and plunge-frozen in liquid ethane using a Vitrobot™ Mark IV (FEI Thermo Scientific). All cryoEM grids were screened on a Glacios™ transmission electron microscope (FEI Thermo Scientific) operated at 200 kV and equipped with a Gatan K2 or K3 Summit direct detector. Automated glacios data collection was carried out using Leginon (48) at a nominal magnification of 36,000× (1.16 Å/pixel). Movies were acquired in counting mode fractionated in 50 frames of 200 ms at 8.5 e-/pixel/sec for a total dose of ˜65e-/Å².

### CryoEM Data Processing:

Multiple datasets were collected for each design and combined early on during processing. Briefly, images were manually curated to remove poor quality acquisitions such as bad ice or large regions of carbon. Dose-weighting and image alignment of all 50 frames was carried out using MotionCor2 (49) with 5×5 patch or with cryosparc v2 patch alignment tool with default parameters. Super-resolution data was binned 2× during alignment. Initial CTF parameters were estimated using CTFfind4 (50). Particle picking was done with a gaussian blob picker and in some cases followed by a template picker. Particles were extensively classified in 2D to remove ice and noisy particles, yielding in some cases relatively few particles. Starting models for all designs were always obtained ab initio, despite clear evidence of the expected design in 2D. FSC curves were generated using cryoSPARC.

### Visualization and Figures

All structural images for figures were generated using PyMOL, Chimera or ChimeraX. Data was processed and figures were plotted using Pandas, MatplotLib, and Seaborn python libraries. Figures were further rendered and assembled using Adobe Illustrator and Inkscape.

TABLE 2
PDB IDs of the closest matches for
structurally-validated HALs (FIG. 2-3).

| | Protomer | | Oligomer | |
|---|---|---|---|---|
| Design | TM-score | PDB | TM-score | Biounit |
| HALC2_062 | 0.69 | 5J1P | 0.59 | 6IU4_1 |
| HALC2_065 | 0.67 | 5W8O | 0.54 | 1XS0_1 |
| HALC2_068 | 0.67 | 4PD6 | 0.57 | 2MFZ_1 |
| HALC3_104 | 0.87 | 7X8V | 0.88 | 5KA5_1 |
| HALC3_109 | 0.78 | 4AIN | 0.69 | 4MOA_3 |
| HALC4_135 | 0.80 | 7RTN | 0.59 | 5VB2_1 |
| HALC4_136 | 0.80 | 1W99 | 0.71 | 7KUY_1 |
| HALC6_220 | 0.65 | 7DPA | 0.51 | 6NYF_1 |
| HALC15-5_262 | 0.65 | 1YRG | 0.46 | 4I0U_1 |
| HALC18-6_265 | 0.65 | 4K17 | 0.49 | 5LNU_1 |
| HALC18-6_278 | 0.65 | 5IRL | 0.49 | 3FEM_1 |
| HALC20-5_308 | 0.59 | 5K7V | 0.45 | 4I0U_1 |
| HALC24-6_316 | 0.69 | 6VFK | 0.44 | 1HB9_1 |
| HALC25-5_341 | 0.59 | 5K7V | 0.45 | 2IUB_2 |
| HALC42-7_351 | 0.58 | 5AWG | 0.41 | 3J26_1 |
| HALC33-3_343 | 0.48 | 4K17 | 0.41 | 1DAB_2 |

TABLE 3
UniRef100 IDs of the best hits for structurally-validated HALs (FIG. 2-3).

| | | | Protomer E- | |
|---|---|---|---|---|
| Design | Repeat_E-value | UniRef100 ID | value | UniRef100 ID |
| HALC2_062 | 3.70E+00 | UPI00131BD06C | 3.70E+00 | UPI00131BD06C |
| HALC2_065 | 5.80E−01 | A0A8I1R8D5 | 5.80E−01 | A0A8I1R8D5 |
| HALC2_068 | 1.40E+00 | A0A6B2M0S8 | 1.40E+00 | A0A6B2M0S8 |
| HALC3_104 | 4.70E−01 | UPI0013B3A05C | 4.70E−01 | UPI0013B3A05C |
| HALC3_109 | 8.20E−01 | UPI000B0DABIF | 8.20E−01 | UPI000B0DAB1F |
| HALC4_135 | 2.80E−01 | A0A3G1RPF3 | 2.80E−01 | A0A3G1RPF3 |
| HALC4_136 | 6.50E+00 | A7ANS2 | 6.50E+00 | A7ANS2 |
| HALC6_220 | 2.00E−02 | A0A434I672 | 2.00E−02 | A0A434I672 |
| HALC15-5_262 | 5.70E−02 | I7LU18 | 3.50E−17 | A0A7S2JY04 |
| HALC18-6_265 | 8.00E−03 | W2S5F8 | 3.17E−16 | A0A7S2JY04 |
| HALC18-6_278 | 5.00E−01 | A0A7E5WBQ0 | 2.99E−08 | A0A819R934 |
| HALC20-5_308 | 9.60E+00 | A0A1F4XIB2 | 1.13E−05 | UPI001CF37084 |
| HALC24-6_316 | 1.00E+01 | UPI0019D624AA | 3.00E−03 | A0A7G8BM39 |
| HALC25-5_341 | 2.60E+01 | A0A6N1YEJ1 | 1.86E−09 | A0A2B4S1A5 |
| HALC33-3_343 | 8.80E−01 | D7MIU3 | 1.62E−35 | A0A2I0HQ60 |
| HALC42-7_351 | 1.40E+01 | A0A7L1D0M5 | 1.35E−14 | B4SHG6 |

TABLE 4
Crystallographic statistics and PDB accession numbers for the structures displayed in FIG. 2.

| | HALC2_062 | | HALC2_065 | | HALC2_068 | | HALC3_104 | |
|---|---|---|---|---|---|---|---|---|
| | PDB: 8D04 | | PDB: 8D03 | | PDB: 8D05 | | PDB: 8D06 | |
| Space group | P 65 | | P 42 | | P 32 2 1 | | P 41 | |
| Cell dimensions | | | | | | | | |
| a, b, c (Å) | 67.9, 67.9, 228.4 | | 50.2, 50.2, 22.1 | | 70.6, 70.6, 31.4 | | 107.5, 107.5, 111.7 | |
| α, β, γ (°) | 90, 90, 120 | | 90, 90, 90 | | 90, 90, 120 | | 90, 90, 90 | |
| Data Collection | | | | | | | | |
| Resolution (Å)* | 56.95-2.11 | (2.19-2.11) | 50.19-2.51 | (2.60-2.51) | 20.39-1.75 | (1.81-1.75) | 76.01-3.40 | (3.52-3.40) |
| Rmerge | 0.067 | (2.197) | 0.311 | (1.853) | 0.447 | (1.368) | 0.076 | (0.641) |
| Rpim | 0.028 | (0.878) | 0.089 | (0.515) | 0.151 | (0.496) | 0.037 | (0.344) |
| Mean I/σ(I) | 16.65 | (1.17) | 2.85 | (0.66) | 8.85 | (1.33) | 14.56 | (2.61) |
| CC 1/2 | 0.996 | (0.559) | 0.987 | (0.336) | 0.95 | (0.566) | 0.999 | (0.748) |
| Completeness (%) | 99.81 | (99.47) | 99.90 | (100) | 98.89 | (89.99) | 99.40 | (99.43) |
| Redundancy | 7.1 | (7.2) | 13.2 | (14.0) | 9.8 | (8.1) | 4.8 | (4.3) |
| Refinement | | | | | | | | |
| No. unique | 34088 | (3405) | 2002 | (193) | 9287 | (819) | 17541 | (1749) |

| | | | | |
|---|---|---|---|---|
| reflections | | | | |
| Rwork/Rfree (%) | 23.6 (32.1)/26.3 (33.4) | 24.2 (41.3)/26.5 (34.8) | 19.0 (27.4)/20.5 (26.2) | 28.4 (35.6)/30.9 (38.3) |
| No. non- | 3210 | 469 | 563 | 6344 |
| hydrogen atoms | | | | |
| Macromolecules | 3210 | 469 | 538 | 6344 |
| Solvent | 0 | 0 | 25 | 0 |
| Ramachandran | 96.52/3.48 | 94.83/5.17 | 98.41/1.59 | 97.33/2.67 |
| favoured/allowed (%) | | | | |
| R.m.s. deviations | | | | |
| Bond lengths (Å) | 0.003 | 0.002 | 0.006 | 0.003 |
| Bond angles (°) | 0.51 | 0.48 | 0.77 | 0.53 |
| B-factors (Å2) | | | | |
| Macromolecules | 76.64 | 74.11 | 35.68 | 139.29 |
| Solvent | | | 42.86 | |

| | HALC3_109 | | HALC4_135 | | HALC4_136 | |
|---|---|---|---|---|---|---|
| | PDB: 8D07 | | PDB: 8D08 | | PDB: 8D09 | |
| Space group | C 1 2 1 | | P 41 21 2 | | C 2 2 21 | |
| Cell dimensions | | | | | | |
| a, b, c (Å) | 136.8, 136.8, 94.2 | | 35..9, 35.9, 438.0 | | 52.8, 77.9, 52.8 | |
| α, β, γ (°) | 90, 129.7, 90 | | 90, 90, 90 | | 90, 90, 90 | |
| Data Collection | | | | | | |
| Resolution (Å)* | 72.61-2.09 | (2.17-2.09) | 54.75-3.30 | (3.41-3.30) | 23.62-1.90 | (1.97-1.90) |
| Rmerge | 0.089 | (0.684) | 0.148 | (0.819) | 0.351 | (0.884) |
| Rpim | 0.050 | (0.400) | 0.060 | (0.328) | 0.102 | (0.244) |
| Mean I/σ(I) | 11.77 | (2.06) | 9.29 | (1.37) | 9.45 | (3.95) |
| CC 1/2 | 0.995 | (0.621) | 0.996 | (0.639) | 0.981 | (0.832) |
| Completeness (%) | 98.51 | (99.27) | 98.67 | (95.19) | 99.52 | (100) |
| Redundancy | 3.8 | (3.9) | 6.8 | (6.4) | 13.1 | (13.6) |
| Refinement | | | | | | |
| No. unique | 20957 | (2047) | 8009 | (435) | 8869 | (875) |

| | | | |
|---|---|---|---|
| reflections | | | |
| Rwork/Rfree (%) | 20.6 (27.0)/26.7 (34.8) | 25.0 (39.8)/29.8 (43.7) | 23.2 (28.2)/25.5 (30.5) |
| No. non- | 3159 | 2208 | 1056 |
| hydrogen atoms | | | |
| Macromolecules | 3159 | 2208 | 1020 |
| Solvent | 0 | 0 | 36 |
| Ramachandran | 99.20/0.80 | 92.58/7.42 | 99.21/0.79 |
| favoured/allowed (%) | | | |
| R.m.s. deviations | | | |
| Bond lengths (Å) | 0.007 | 0.011 | 0.014 |
| Bond angles (°) | 0.92 | 1.37 | 1.47 |
| B-factors (Å2) | | | |
| Macromolecules | 54.51 | 134.25 | 37.12 |
| Solvent | | | 44.17 |

*Statistics for the highest-resolution shell are shown in parentheses

### Supplementary References

1. H. Garcia-Seisdedos, C. Empereur-Mot, N. Elad, E. D. Levy, Proteins evolve on the edge of supramolecular self-assembly. Nature. 548, 244-247 (2017).

2. I. G. Johnston, K. Dingle, S. F. Greenbury, C. Q. Camargo, J. P. K. Doye, S. E. Ahnert, A. A. Louis, Symmetry and simplicity spontaneously emerge from the algorithmic nature of evolution. Proc. Natl. Acad. Sci. 119, e2113883119 (2022).

3. S. E. Ahnert, J. A. Marsh, H. Hernandez, C. V. Robinson, S. A. Teichmann, Principles of assembly reveal a periodic table of protein complexes. Science. 350, aaa2245 (2015).

4. wwPDB consortium, Protein Data Bank: the single global archive for 3D macromolecular structure data. Nucleic Acids Res. 47, D520—D528 (2019).

5. D. S. Goodsell, A. J. Olson, Structural Symmetry and Protein Function. Annu. Rev. Biophys. Biomol. Struct. 29, 105-153 (2000).

6. T. Handel, W. F. DeGrado, De novo design of a Zn2+-binding protein. J. Am. Chem. Soc. 112, 6710-6711 (1990).

7. P. B. Harbury, J. J. Plecs, B. Tidor, T. Alber, P. S. Kim, High-Resolution Protein Design with Backbone Freedom. Science. 282, 1462-1467 (1998).

8. J. A. Fallas, G. Ueda, W. Sheffler, V. Nguyen, D. E. McNamara, B. Sankaran, J. H. Pereira, F. Parmeggiani, T. J. Brunette, D. Cascio, T. R. Yeates, P. Zwart, D. Baker, Computational design of self-assembling cyclic protein homo-oligomers. Nat. Chem. 9, 353-360 (2017).

9. A. R. Thomson, C. W. Wood, A. J. Burton, G. J. Bartlett, R. B. Sessions, R. L. Brady, D. N. Woolfson, Computational design of water-soluble α-helical barrels. Science. 346, 485-488 (2014).

10. P.-S. Huang, K. Feldmeier, F. Parmeggiani, D. A. Fernandez Velasco, B. Hocker, D. Baker, De novo design of a four-fold symmetric TIM-barrel protein with atomic-level accuracy. Nat. Chem. Biol. 12, 29-34 (2016).

11. P.-S. Huang, G. Oberdorfer, C. Xu, X. Y. Pei, B. L. Nannenga, J. M. Rogers, F. DiMaio, T. Gonen, B. Luisi, D. Baker, High thermodynamic stability of parametrically designed helical bundles. Science. 346, 481-485 (2014).

12. S. E. Boyken, Z. Chen, B. Groves, R. A. Langan, G. Oberdorfer, A. Ford, J. M. Gilmore, C. Xu, F. DiMaio, J. H. Pereira, B. Sankaran, G. Seelig, P. H. Zwart, D. Baker, De novo design of protein homo-oligomers with modular hydrogen-bond network-mediated specificity. Science. 352, 680-687 (2016).

13. J. B. Bale, S. Gonen, Y. Liu, W. Sheffler, D. Ellis, C. Thomas, D. Cascio, T. O. Yeates, T. Gonen, N. P. King, D. Baker, Accurate design of megadalton-scale two-component icosahedral protein complexes. Science. 353, 389-394 (2016).

14. I. Vulovic, et al., Generation of ordered protein assemblies using rigid three-body fusion. Proc. Natl. Acad. Sci. 118, e2015037118 (2021).

15. Y. Hsia, et al., Design of multi-scale protein complexes by hierarchical building block fusion. Nat. Commun. 12, 2294 (2021).

16. C. E. Correnti, et al., Engineering and functionalization of large circular tandem repeat protein nanoparticles. Nat. Struct. Mol. Biol. 27, 342-350 (2020).

17. D. D. Sahtoe, F. Praetorius, A. Courbet, Y. Hsia, B. I. M. Wicky, N. I. Edman, L. M. Miller, B. J. R. Timmermans, J. Decarreau, H. M. Morris, A. Kang, A. K. Bera, D. Baker, Reconfigurable asymmetric protein assemblies through implicit negative design. Science. 375, eabj7662 (2022).

18. I. Anishchenko, S. J. Pellock, T. M. Chidyausiku, T. A. Ramelot, S. Ovchinnikov, J. Hao, K. Bafna, C. Norn, A. Kang, A. K. Bera, F. DiMaio, L. Carter, C. M. Chow, G. T. Montelione, D. Baker, De novo protein design by deep network hallucination. Nature. 600, 547-552 (2021).

19. M. Jendrusch, J. O. Korbel, S. K. Sadiq, AlphaDesign: A de novo protein design framework based on AlphaFold (2021), p. 2021.10.11.463937, doi:10.1101/2021.10.11.463937.

20. L. Moffat, J. G. Greener, D. T. Jones, Using AlphaFold for Rapid and Accurate Fixed Backbone Protein Design (2021), p. 2021.08.24.457549, doi:10.1101/2021.08.24.457549.

21. J. Wang, S. Lisanza, D. Juergens, D. Tischer, I. Anishchenko, M. Baek, J. L. Watson, J. H. Chun, L. F. Milles, J. Dauparas, M. Exposit, W. Yang, A. Saragovi, S. Ovchinnikov, D. Baker, Deep learning methods for designing proteins scaffolding functional sites (2021), p. 2021.11.10.468128, doi:10.1101/2021.11.10.468128.

22. S. Ovchinnikov, P.-S. Huang, Structure-based protein design with deep learning. Curr. Opin. Chem. Biol. 65, 136-144 (2021).

23. C. Norn, et al., Protein sequence design by conformational landscape optimization. Proc. Natl. Acad. Sci. 118, e2017228118 (2021).

24. N. Anand, R. Eguchi, I. I. Mathews, C. P. Perez, A. Derry, R. B. Altman, P.-S. Huang, Protein sequence design with a learned potential. Nat. Commun. 13, 746 (2022).

25. J. Jumper, et al., Highly accurate protein structure prediction with AlphaFold. Nature. 596, 583-589 (2021).

26. J. Xu, Y. Zhang, How significant is a protein structure similarity with TM-score=0.5 Bioinformatics. 26, 889-895 (2010).

27. Inceptionism: Going Deeper into Neural Networks. Google AI Blog, (ai.googleblog.com/2015/06/inceptionism-going-deeper-into-neural).

28. A. Nguyen, J. Yosinski, J. Clune, Deep Neural Networks are Easily Fooled: High Confidence Predictions for Unrecognizable Images (2015), (arxiv.org/abs/1412.1897).

29. K. Simonyan, A. Vedaldi, A. Zisserman, Deep Inside Convolutional Networks: Visualising Image Classification Models and Saliency Maps (2014), (arxiv. org/ab s/1312.6034).

30. M. Baek, et al., Accurate prediction of protein structures and interactions using a three-track neural network. Science. 373, 871-876 (2021).

31. B. Kobe, J. Deisenhofer, The leucine-rich repeat: a versatile binding motif. Trends Biochem. Sci. 19, 415-421 (1994).

32. P. Guerra, M. Gonzalez-Alamos, A. Llauro, A. Casafias, J. Querol-Audi, P. J. de Pablo, N. Verdaguer, Symmetry disruption commits vault particles to disassembly. Sci. Adv. 8, eabj7795 (2022).

33. A. Courbet, et al., Computational design of mechanically coupled axle-rotor protein assemblies. Science. 376, 383-390 (2022).

34. Y. Zhang, J. Skolnick, TM-align: a protein structure alignment algorithm based on the TM-score. Nucleic Acids Res. 33, 2302-2309 (2005).

35. S. Mukherjee, Y. Zhang, MM-align: a quick algorithm for aligning multiple-chain protein complex structures using iterative dynamic programming. Nucleic Acids Res. 37, e83 (2009).

36. B. Dang, M. Mravic, H. Hu, N. Schmidt, B. Mensa, W. F. DeGrado, SNAC-tag for sequence-specific chemical protein cleavage. Nat. Methods. 16, 319-322 (2019).

37. W. Kabsch, XDS. Acta Crystallogr. D Biol. Crystallogr. 66, 125-132 (2010).

38. M. D. Winn, et al., Overview of the CCP4 suite and current developments. Acta Crystallogr. D Biol. Crystallogr. 67, 235-242 (2011).

39. A. J. McCoy, R. W. Grosse-Kunstleve, P. D. Adams, M. D. Winn, L. C. Storoni, R. J. Read, Phaser crystallographic software. J. Appl. Crystallogr. 40, 658-674 (2007).

40. P. Emsley, K. Cowtan, Coot: model-building tools for molecular graphics. Acta Crystallogr. D Biol. Crystallogr. 60, 2126-2132 (2004).

41. P. D. Adams, et al., PHENIX: a comprehensive Python-based system for macromolecular structure solution. Acta Crystallogr. D Biol. Crystallo . 66, 213-221 (2010).

42. G. N. Murshudov, A. A. Vagin, E. J. Dodson, Refinement of Macromolecular Structures by the Maximum-Likelihood Method. Acta Crystallogr. D Biol. Crystallogr. 53, 240-255 (1997).

43. C. J. Williams, Jet al., MolProbity: More and better reference data for improved all-atom structure validation. Protein Sci. 27, 293-315 (2018).

44. B. L. Nannenga, M. G. Iadanza, B. S. Vollmar, T. Gonen, Curr. Protoc. Protein Sci., in press, doi:10.1002/0471140864.ps1715s72.

45. T. Grant, A. Rohou, N. Grigorieff, cisTEM, user-friendly software for single-particle image processing. eLife. 7, e35383 (2018).

46. A. Punjani, J. L. Rubinstein, D. J. Fleet, M. A. Brubaker, cryoSPARC: algorithms for rapid unsupervised cryo-EM structure determination. Nat. Methods. 14, 290-296 (2017).

47. A. Punjani, D. J. Fleet, 3D variability analysis: Resolving continuous flexibility and discrete heterogeneity from single particle cryo-EM. J. Struct. Biol. 213, 107702 (2021).

48. B. Carragher, N. Kisseberth, D. Kriegman, R. A. Milligan, C. S. Potter, J. Pulokas, A. Reilein, Leginon: An Automated System for Acquisition of Images from Vitreous Ice Specimens. J. Struct. Biol. 132, 33-45 (2000).

49. S. Q. Zheng, E. Palovcak, J.-P. Armache, K. A. Verba, Y. Cheng, D. A. Agard, MotionCor2: anisotropic correction of beam-induced motion for improved cryo-electron microscopy. Nat. Methods. 14, 331-332 (2017).

50. A. Rohou, N. Grigorieff, CTFFIND4: Fast and accurate defocus estimation from electron micrographs. J. Struct. Biol. 192, 216-221 (2015).

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

We claim:

1. A polypeptide comprising an amino acid sequence at least 50% identical to the amino acid sequence selected from the group consisting of SEQ ID NOS:1-38, wherein any N-terminal amino acid is optional and may be present or may be deleted.

2. The polypeptide of claim 1, comprising an amino acid sequence at least 75% identical to the amino acid sequence selected from the group consisting of SEQ ID NOS:1-38, wherein any N-terminal amino acid is optional and may be present or may be deleted.

3. The polypeptide of claim 1, comprising an amino acid sequence at least 90% identical to the amino acid sequence selected from the group consisting of SEQ ID NOS:1-38, wherein any N-terminal amino acid is optional and may be present or may be deleted.

4. The polypeptide of claim 1, wherein at least 50% of substitutions relative to the reference amino acid sequence are at surface residues as defined in Table 1.

5. The polypeptide of claim 1, wherein at least 50% of core residues, as defined in Table 1 are maintained as in the reference amino acid sequence.

6. The polypeptide of claim 1, wherein substitutions relative to the reference sequence are conservative amino acid substitutions.

7. The polypeptide of claim 1, further comprising one or more functional domains.

8. A cyclic homo-oligomer, comprising one or a plurality of the polypeptides of claim 1.

9. The cyclic homo-oligomer of claim 8, comprising a plurality of identical polypeptides of claim 1

10. The cyclic homo-oligomer of claim 8, wherein the cyclic homo-oligomer has a symmetry as listed in Table 1.

11. The cyclic homo-oligomer of claim 8, wherein the homo-oligomer has a pseudosymmetry (number of chains) as listed in Table 1.

12. The cyclic homo-oligomer of claim 8, comprising an amino acid sequence at least 50% identical to the amino acid sequence selected from SEQ ID NO:1-5 and 39-71.

13. The cyclic homo-oligomer of claim 8, wherein the cyclic homo-oligomer maintains its secondary structure at temperatures up to 95° C.

14. The cyclic homo-oligomer of claim 8, wherein the cyclic homo-oligomer has a size along its largest dimension of between about 5 and about 16 nm.

15. A nucleic acid encoding the polypeptide of claim 1.

16. An expression vector comprising the nucleic acid of claim 15 operatively linked to a suitable control sequence.

17. A host cell comprising the expression vector of claim 16.

18. A method for generating an immune response, comprising administering to a subject in need thereof a cyclic homo-oligomer according claim 8, wherein the cyclic homo-oligomer comprises an antigen scaffold on a surface of the cyclic homo-oligomer, in an amount effective to generate an immune response against the antigen in the subject.


SEQUENCE LISTING (71 sequences)

SEQ ID NO: 1 - protein, 65 aa, synthetic construct
  REGION 1..65: note = synthetic peptide
MIVSLEKHPGGVHIITLSSEENLENFVKELKKLGAEVERLPEPNTVRVRAPEEVVEEALKNTKFK

SEQ ID NO: 2 - protein, 65 aa, synthetic construct
  REGION 1..65: note = synthetic peptide
NEKEFLLQLKEELDKDPSEENVLSLIKTLNEEQKKILEEIKKKYPNLPLSKIFELLIDELLERLE

SEQ ID NO: 3 - protein, 65 aa, synthetic construct
  REGION 1..65: note = synthetic peptide
DKIAFFKRLKEELEKDPSDENVEKLIETLNEEEKKILEEIKKEYPNEPLSEIFYKLIEKLLELSE

SEQ ID NO: 4 - protein, 65 aa, synthetic construct
  REGION 1..65: note = synthetic peptide
MLSPEELLEKLKKYLKEKYNVVVPEERIVSVEATDSSVKLTWSRGDGREGTAYYSDEGEVRVEDP

SEQ ID NO: 5 - protein, 65 aa, synthetic construct
  REGION 1..65: note = synthetic peptide
MLTPEELLERLRRHLEEEHGVVVPEERILSVEATPTEVTLTWSRGDGRTGTARYTSDGRFEVEDP

SEQ ID NO: 6 - protein, 65 aa, synthetic construct
  REGION 1..65: note = synthetic peptide
MVKPITEEDVREAATAASPDYEVGEAKLIDEENNLWFVTLYKGDQKIYALIEDKNGEFTVHQIEL

SEQ ID NO: 7 - protein, 65 aa, synthetic construct
  REGION 1..65: note = synthetic peptide
MARVEYSYEKLNDTHYKLKLKVTYEYRKSPEARRLAEDLVQAFVDALSSLPFITVEYEVEEVEVE

SEQ ID NO: 8 - protein, 65 aa, synthetic construct
  REGION 1..65: note = synthetic peptide
MKVYEFPYPETGKKIIVIQGEKNIVIVVGNTAVVYYEGKWTYKENVTEEDIEKAKTEEGAKELAK

SEQ ID NO: 9 - protein, 65 aa, synthetic construct
  REGION 1..65: note = synthetic peptide
SKLKEQEELIDEISEKAKEFLLEIKEKYPGELSEERYPGRVVLTYVNEEKGFSITVTIELLNKEK

SEQ ID NO: 10 - protein, 65 aa, synthetic construct
  REGION 1..65: note = synthetic peptide
SEEEKPIVIDLNKTIERDGRKVKLVRATITVDPETNTITIDIEYEGGPITKEDLLEAFKLAASKL

SEQ ID NO: 11 - protein, 65 aa, synthetic construct
  REGION 1..65: note = synthetic peptide
DKLVRVLSSSMIYYAERMTKGSTDPSDYDKALDDFYNYFLEQPFVDKETLEKAYELARKRLEELL

SEQ ID NO: 12 - protein, 65 aa, synthetic construct
  REGION 1..65: note = synthetic peptide
MIKVPEDLERIGRELRARGLDTKRLLEEGPKLYPELSIPDLMAIALYDHLNLDPEFLYRLLQQSR

SEQ ID NO: 13 - protein, 65 aa, synthetic construct
  REGION 1..65: note = synthetic peptide
LEELKERVEQLEKRLSVVESTLTHLLTTFSDETLKWIYDNTRADPSVDKETLDEFWKRVEEEKKK

SEQ ID NO: 14 - protein, 65 aa, synthetic construct
  REGION 1..65: note = synthetic peptide
KRIDEIESKLKHLEEFTTHLIKLMETMLELLKLVSDGKSDSEEYKELLEKAEEYLKQATEAAKKI

SEQ ID NO: 15 - protein, 65 aa, synthetic construct
  REGION 1..65: note = synthetic peptide
MEPEELERLRELYEVFKDKLDEPIGLYLLTLLAIYDPERREEYLEKLRDIFEKQGETDIAERLKE

SEQ ID NO: 16 - protein, 65 aa, synthetic construct
  REGION 1..65: note = synthetic peptide
REEIEEAVKEAELKVLAIVLVALRSVSHYEPLSRLYESFLDALKKALSEEELKEVEKEAERIEKK

SEQ ID NO: 17 - protein, 65 aa, synthetic construct
  REGION 1..65: note = synthetic peptide
LEQILEELTELLERVDEIPLREALKRMLELLVRVTQELKEVKDKVESLEKHLEELDKRVEEIEKK

SEQ ID NO: 18 - protein, 65 aa, synthetic construct
  REGION 1..65: note = synthetic peptide
MAKLLPGLSEEEKRLTDILDKLLPGLEVLDVLREDGLVVFLARHGDHLLVASFTRFKDPELQSKV

SEQ ID NO: 19 - protein, 65 aa, synthetic construct
  REGION 1..65: note = synthetic peptide
MTKLVEYHYDEETQLLYIKLQLNENEYLVLFLYSKEDEESLKKLKELEEEAASDPSLHLVKGFFK

SEQ ID NO: 20 - protein, 65 aa, synthetic construct
  REGION 1..65: note = synthetic peptide
VDEKEVKERFEEIESRLEELESKVREVEKKVEEVKKESDEKIDQLKTEFETKYNQINNEINTLKN

SEQ ID NO: 21 - protein, 65 aa, synthetic construct
  REGION 1..65: note = synthetic peptide
MTRLEQLLAQGVDPFEVLREKIEKLKEIWKKYEEAKGEEKERYRDELLKLMMEVLELMVELLSRR

SEQ ID NO: 22 - protein, 65 aa, synthetic construct
  REGION 1..65: note = synthetic peptide
MEKFKEQLLEEVKKIVLETMTKVMEHLEKWFVTLAEIIITKSEEKLEELKETMEKSIEELRKEAE

SEQ ID NO: 23 - protein, 65 aa, synthetic construct
  REGION 1..65: note = synthetic peptide
MSPYKKAIEITKRLLELLLSNPELAKKNLGGIATLISLLALISALDGTLDEKDIEPYIKKLEESL

SEQ ID NO: 24 - protein, 65 aa, synthetic construct
  REGION 1..65: note = synthetic peptide
MEEVVLTSHNELHKKLDEVHDKIMSKLDEIHEKLDEIISKLDEIESKLHEILNIVKEIKEILEKK

SEQ ID NO: 25 - protein, 65 aa, synthetic construct
  REGION 1..65: note = synthetic peptide
SSLKEWLERWREKLVEAVKGTPEEEKVEKYLDLALESLEEMPDKKLAERIASRLFTEAVKTVVEA

SEQ ID NO: 26 - protein, 65 aa, synthetic construct
  REGION 1..65: note = synthetic peptide
LLLEVMEKVFDEEQLKLIKEAAEREGNSPVVISSIATLLLLERIEKIVKEIHDEVKKNNEKQEKK

SEQ ID NO: 27 - protein, 65 aa, synthetic construct
  REGION 1..65: note = synthetic peptide
RPRPPIRLEVLIEADLSDPDSLLRAIEEAERTLERLERDLPPEVLERFRPHLRLEILLKKDIKPE

SEQ ID NO: 28 - protein, 65 aa, synthetic construct
  REGION 1..65: note = synthetic peptide
MDPKELEREALKNIIKLPKLIQDFKDSVMKELNKIIELLEERRREIDEPLLPIIRKLQEELQKKE

SEQ ID NO: 29 - protein, 65 aa, synthetic construct
  REGION 1..65: note = synthetic peptide
RKIPYDPNRDLYITITLTVRNNPDQKSFLQSIDLLIKLLEQGYRVTINLVDFNTKEEKEQALQQL

SEQ ID NO: 30 - protein, 210 aa, synthetic construct
  REGION 1..210: note = synthetic peptide
DVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFN

SEQ ID NO: 31 - protein, 210 aa, synthetic construct
  REGION 1..210: note = synthetic peptide
NIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTIS

SEQ ID NO: 32 - protein, 517 aa, synthetic construct
  REGION 1..517: note = synthetic peptide
PSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGL

SEQ ID NO: 33 - protein, 144 aa, synthetic construct
  REGION 1..144: note = synthetic peptide
PPIPPPSFKLEISPAFLELVQLVIDLHPNDEEVRKELIENLISRIGKSDNVPPETISLDISEAALELFEWIFEKFPDDEDVHRRLIESFINKRKFSSSSPLDTPSLDISERFIELVKYILEKYPEDEEIKQKLIDSLLNLLGSY

SEQ ID NO: 34 - protein, 168 aa, synthetic construct
  REGION 1..168: note = synthetic peptide
SKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSP

SEQ ID NO: 35 - protein, 212 aa, synthetic construct
  REGION 1..212: note = synthetic peptide
SLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTG

SEQ ID NO: 36 - protein, 210 aa, synthetic construct
  REGION 1..210: note = synthetic peptide
EGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSP

SEQ ID NO: 37 - protein, 210 aa, synthetic construct
  REGION 1..210: note = synthetic peptide
GERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKIL

SEQ ID NO: 38 - protein, 240 aa, synthetic construct
  REGION 1..240: note = synthetic peptide
PSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVST

SEQ ID NO: 39 - protein, 130 aa, synthetic construct
  REGION 1..130: note = synthetic peptide
MVKPITEEDVREAATAASPDYEVGEAKLIDEENNLWFVTLYKGDQKIYALIEDKNGEFTVHQIELMVKPITEEDVREAATAASPDYEVGEAKLIDEENNLWFVTLYKGDQKIYALIEDKNGEFTVHQIEL

SEQ ID NO: 40 - protein, 130 aa, synthetic construct
  REGION 1..130: note = synthetic peptide
MARVEYSYEKLNDTHYKLKLKVTYEYRKSPEARRLAEDLVQAFVDALSSLPFITVEYEVEEVEVEMARVEYSYEKLNDTHYKLKLKVTYEYRKSPEARRLAEDLVQAFVDALSSLPFITVEYEVEEVEVE

SEQ ID NO: 41 - protein, 130 aa, synthetic construct
  REGION 1..130: note = synthetic peptide
MKVYEFPYPETGKKIIVIQGEKNIVIVVGNTAVVYYEGKWTYKENVTEEDIEKAKTEEGAKELAKMKVYEFPYPETGKKIIVIQGEKNIVIVVGNTAVVYYEGKWTYKENVTEEDIEKAKTEEGAKELAK

SEQ ID NO: 42 - protein, 130 aa, synthetic construct
  REGION 1..130: note = synthetic peptide
SKLKEQEELIDEISEKAKEFLLEIKEKYPGELSEERYPGRVVLTYVNEEKGFSITVTIELLNKEKSKLKEQEELIDEISEKAKEFLLEIKEKYPGELSEERYPGRVVLTYVNEEKGFSITVTIELLNKEK

SEQ ID NO: 43 - protein, 130 aa, synthetic construct
  REGION 1..130: note = synthetic peptide
SEEEKPIVIDLNKTIERDGRKVKLVRATITVDPETNTITIDIEYEGGPITKEDLLEAFKLAASKLSEEEKPIVIDLNKTIERDGRKVKLVRATITVDPETNTITIDIEYEGGPITKEDLLEAFKLAASKL

SEQ ID NO: 44 - protein, 130 aa, synthetic construct
  REGION 1..130: note = synthetic peptide
DKLVRVLSSSMIYYAERMTKGSTDPSDYDKALDDFYNYFLEQPFVDKETLEKAYELARKRLEELLDKLVRVLSSSMIYYAERMTKGSTDPSDYDKALDDFYNYFLEQPFVDKETLEKAYELARKRLEELL

SEQ ID NO: 45 - protein, 130 aa, synthetic construct
  REGION 1..130: note = synthetic peptide
MIKVPEDLERIGRELRARGLDTKRLLEEGPKLYPELSIPDLMAIALYDHLNLDPEFLYRLLQQSRMIKVPEDLERIGRELRARGLDTKRLLEEGPKLYPELSIPDLMAIALYDHLNLDPEFLYRLLQQSR

SEQ ID NO: 46 - protein, 195 aa, synthetic construct
  REGION 1..195: note = synthetic peptide
LEELKERVEQLEKRLSVVESTLTHLLTTFSDETLKWIYDNTRADPSVDKETLDEFWKRVEEEKKKLEELKERVEQLEKRLSVVESTLTHLLTTFSDETLKWIYDNTRADPSVDKETLDEFWKRVEEEKKKLEELKERVEQLEKRLSVVESTLTHLLTTFSDETLKWIYDNTRADPSVDKETLDEFWKRVEEEKKK

SEQ ID NO: 47 - protein, 195 aa, synthetic construct
  REGION 1..195: note = synthetic peptide
KRIDEIESKLKHLEEFTTHLIKLMETMLELLKLVSDGKSDSEEYKELLEKAEEYLKQATEAAKKIKRIDEIESKLKHLEEFTTHLIKLMETMLELLKLVSDGKSDSEEYKELLEKAEEYLKQATEAAKKIKRIDEIESKLKHLEEFTTHLIKLMETMLELLKLVSDGKSDSEEYKELLEKAEEYLKQATEAAKKI

SEQ ID NO: 48 - protein, 195 aa, synthetic construct
  REGION 1..195: note = synthetic peptide
MEPEELERLRELYEVFKDKLDEPIGLYLLTLLAIYDPERREEYLEKLRDIFEKQGETDIAERLKEMEPEELERLRELYEVFKDKLDEPIGLYLLTLLAIYDPERREEYLEKLRDIFEKQGETDIAERLKEMEPEELERLRELYEVFKDKLDEPIGLYLLTLLAIYDPERREEYLEKLRDIFEKQGETDIAERLKE

SEQ ID NO: 49 - protein, 195 aa, synthetic construct
  REGION 1..195: note = synthetic peptide
REEIEEAVKEAELKVLAIVLVALRSVSHYEPLSRLYESFLDALKKALSEEELKEVEKEAERIEKKREEIEEAVKEAELKVLAIVLVALRSVSHYEPLSRLYESFLDALKKALSEEELKEVEKEAERIEKKREEIEEAVKEAELKVLAIVLVALRSVSHYEPLSRLYESFLDALKKALSEEELKEVEKEAERIEKK

SEQ ID NO: 50 - protein, 195 aa, synthetic construct
  REGION 1..195: note = synthetic peptide
LEQILEELTELLERVDEIPLREALKRMLELLVRVTQELKEVKDKVESLEKHLEELDKRVEEIEKKLEQILEELTELLERVDEIPLREALKRMLELLVRVTQELKEVKDKVESLEKHLEELDKRVEEIEKKLEQILEELTELLERVDEIPLREALKRMLELLVRVTQELKEVKDKVESLEKHLEELDKRVEEIEKK

SEQ ID NO: 51 - protein, 195 aa, synthetic construct
  REGION 1..195: note = synthetic peptide
MAKLLPGLSEEEKRLTDILDKLLPGLEVLDVLREDGLVVFLARHGDHLLVASFTRFKDPELQSKVMAKLLPGLSEEEKRLTDILDKLLPGLEVLDVLREDGLVVFLARHGDHLLVASFTRFKDPELQSKVMAKLLPGLSEEEKRLTDILDKLLPGLEVLDVLREDGLVVFLARHGDHLLVASFTRFKDPELQSKV

SEQ ID NO: 52 - protein, 195 aa, synthetic construct
  REGION 1..195: note = synthetic peptide
MTKLVEYHYDEETQLLYIKLQLNENEYLVLFLYSKEDEESLKKLKELEEEAASDPSLHLVKGFFKMTKLVEYHYDEETQLLYIKLQLNENEYLVLFLYSKEDEESLKKLKELEEEAASDPSLHLVKGFFKMTKLVEYHYDEETQLLYIKLQLNENEYLVLFLYSKEDEESLKKLKELEEEAASDPSLHLVKGFFK

SEQ ID NO: 53 - protein, 195 aa, synthetic construct
  REGION 1..195: note = synthetic peptide
VDEKEVKERFEEIESRLEELESKVREVEKKVEEVKKESDEKIDQLKTEFETKYNQINNEINTLKNVDEKEVKERFEEIESRLEELESKVREVEKKVEEVKKESDEKIDQLKTEFETKYNQINNEINTLKNVDEKEVKERFEEIESRLEELESKVREVEKKVEEVKKESDEKIDQLKTEFETKYNQINNEINTLKN

SEQ ID NO: 54 - protein, 195 aa, synthetic construct
  REGION 1..195: note = synthetic peptide
MTRLEQLLAQGVDPFEVLREKIEKLKEIWKKYEEAKGEEKERYRDELLKLMMEVLELMVELLSRRMTRLEQLLAQGVDPFEVLREKIEKLKEIWKKYEEAKGEEKERYRDELLKLMMEVLELMVELLSRRMTRLEQLLAQGVDPFEVLREKIEKLKEIWKKYEEAKGEEKERYRDELLKLMMEVLELMVELLSRR

SEQ ID NO: 55 - protein, 260 aa, synthetic construct
  REGION 1..260: note = synthetic peptide
MEKFKEQLLEEVKKIVLETMTKVMEHLEKWFVTLAEIIITKSEEKLEELKETMEKSIEELRKEAEMEKFKEQLLEEVKKIVLETMTKVMEHLEKWFVTLAEIIITKSEEKLEELKETMEKSIEELRKEAEMEKFKEQLLEEVKKIVLETMTKVMEHLEKWFVTLAEIIITKSEEKLEELKETMEKSIEELRKEAEMEKFKEQLLEEVKKIVLETMTKVMEHLEKWFVTLAEIIITKSEEKLEELKETMEKSIEELRKEAE

SEQ ID NO: 56 - protein, 260 aa, synthetic construct
  REGION 1..260: note = synthetic peptide
MSPYKKAIEITKRLLELLLSNPELAKKNLGGIATLISLLALISALDGTLDEKDIEPYIKKLEESLMSPYKKAIEITKRLLELLLSNPELAKKNLGGIATLISLLALISALDGTLDEKDIEPYIKKLEESLMSPYKKAIEITKRLLELLLSNPELAKKNLGGIATLISLLALISALDGTLDEKDIEPYIKKLEESLMSPYKKAIEITKRLLELLLSNPELAKKNLGGIATLISLLALISALDGTLDEKDIEPYIKKLEESL

SEQ ID NO: 57 - protein, 260 aa, synthetic construct
  REGION 1..260: note = synthetic peptide
MEEVVLTSHNELHKKLDEVHDKIMSKLDEIHEKLDEIISKLDEIESKLHEILNIVKEIKEILEKKMEEVVLTSHNELHKKLDEVHDKIMSKLDEIHEKLDEIISKLDEIESKLHEILNIVKEIKEILEKKMEEVVLTSHNELHKKLDEVHDKIMSKLDEIHEKLDEIISKLDEIESKLHEILNIVKEIKEILEKKMEEVVLTSHNELHKKLDEVHDKIMSKLDEIHEKLDEIISKLDEIESKLHEILNIVKEIKEILEKK

SEQ ID NO: 58 - protein, 325 aa, synthetic construct
  REGION 1..325: note = synthetic peptide
SSLKEWLERWREKLVEAVKGTPEEEKVEKYLDLALESLEEMPDKKLAERIASRLFTEAVKTVVEASSLKEWLERWREKLVEAVKGTPEEEKVEKYLDLALESLEEMPDKKLAERIASRLFTEAVKTVVEASSLKEWLERWREKLVEAVKGTPEEEKVEKYLDLALESLEEMPDKKLAERIASRLFTEAVKTVVEASSLKEWLERWREKLVEAVKGTPEEEKVEKYLDLALESLEEMPDKKLAERIASRLFTEAVKTVVEASSLKEWLERWREKLVEAVKGTPEEEKVEKYLDLALESLEEMPDKKLAERIASRLFTEAVKTVVEA

SEQ ID NO: 59 - protein, 325 aa, synthetic construct
  REGION 1..325: note = synthetic peptide
LLLEVMEKVFDEEQLKLIKEAAEREGNSPVVISSIATLLLLERIEKIVKEIHDEVKKNNEKQEKKLLLEVMEKVFDEEQLKLIKEAAEREGNSPVVISSIATLLLLERIEKIVKEIHDEVKKNNEKQEKKLLLEVMEKVFDEEQLKLIKEAAEREGNSPVVISSIATLLLLERIEKIVKEIHDEVKKNNEKQEKKLLLEVMEKVFDEEQLKLIKEAAEREGNSPVVISSIATLLLLERIEKIVKEIHDEVKKNNEKQEKKLLLEVMEKVFDEEQLKLIKEAAEREGNSPVVISSIATLLLLERIEKIVKEIHDEVKKNNEKQEKK

SEQ ID NO: 60 - protein, 325 aa, synthetic construct
  REGION 1..325: note = synthetic peptide
RPRPPIRLEVLIEADLSDPDSLLRAIEEAERTLERLERDLPPEVLERFRPHLRLEILLKKDIKPERPRPPIRLEVLIEADLSDPDSLLRAIEEAERTLERLERDLPPEVLERFRPHLRLEILLKKDIKPERPRPPIRLEVLIEADLSDPDSLLRAIEEAERTLERLERDLPPEVLERFRPHLRLEILLKKDIKPERPRPPIRLEVLIEADLSDPDSLLRAIEEAERTLERLERDLPPEVLERFRPHLRLEILLKKDIKPERPRPPIRLEVLIEADLSDPDSLLRAIEEAERTLERLERDLPPEVLERFRPHLRLEILLKKDIKPE

SEQ ID NO: 61 - protein, 325 aa, synthetic construct
  REGION 1..325: note = synthetic peptide
MDPKELEREALKNIIKLPKLIQDFKDSVMKELNKIIELLEERRREIDEPLLPIIRKLQEELQKKEMDPKELEREALKNIIKLPKLIQDFKDSVMKELNKIIELLEERRREIDEPLLPIIRKLQEELQKKEMDPKELEREALKNIIKLPKLIQDFKDSVMKELNKIIELLEERRREIDEPLLPIIRKLQEELQKKEMDPKELEREALKNIIKLPKLIQDFKDSVMKELNKIIELLEERRREIDEPLLPIIRKLQEELQKKEMDPKELEREALKNIIKLPKLIQDFKDSVMKELNKIIELLEERRREIDEPLLPIIRKLQEELQKKE

SEQ ID NO: 62 - protein, 390 aa, synthetic construct
  REGION 1..390: note = synthetic peptide
RKIPYDPNRDLYITITLTVRNNPDQKSFLQSIDLLIKLLEQGYRVTINLVDFNTKEEKEQALQQLRKIPYDPNRDLYITITLTVRNNPDQKSFLQSIDLLIKLLEQGYRVTINLVDFNTKEEKEQALQQLRKIPYDPNRDLYITITLTVRNNPDQKSFLQSIDLLIKLLEQGYRVTINLVDFNTKEEKEQALQQLRKIPYDPNRDLYITITLTVRNNPDQKSFLQSIDLLIKLLEQGYRVTINLVDFNTKEEKEQALQQLRKIPYDPNRDLYITITLTVRNNPDQKSFLQSIDLLIKLLEQGYRVTINLVDFNTKEEKEQALQQLRKIPYDPNRDLYITITLTVRNNPDQKSFLQSIDLLIKLLEQGYRVTINLVDFNTKEEKEQALQQL

SEQ ID NO: 63 - protein, 1050 aa, synthetic construct
  REGION 1..1050: note = synthetic peptide
DVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFNDVPLTDPKNLNEFLYALGEGLKGMKNLKKLTLTFPSNPLTIPGDISEGFRELGEGLKGMKNLEELTVTFN

SEQ ID NO: 64 - protein, 1260 aa, synthetic construct
  REGION 1..1260: note = synthetic peptide
NIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTISNIKIPNPKDLSELLKKLGEGLKGLPNLKTLTLTLSNIELPEDADLSPGAEGLGEGLKGLPNLETLTFTIS

SEQ ID NO: 65 - protein, 1551 aa, synthetic construct
  REGION 1..1551: note = synthetic peptide
PSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGLPSNWPEVAKYFDLGKALKPIGEGLQNLKNLKHLDLSFSFSLELYPGL

SEQ ID NO: 66 - protein, 864 aa, synthetic construct
  REGION 1..864: note = synthetic peptide
PPIPPPSFKLEISPAFLELVQLVIDLHPNDEEVRKELIENLISRIGKSDNVPPETISLDISEAALELFEWIFEKFPDDEDVHRRLIESFINKRKFSSSSPLDTPSLDISERFIELVKYILEKYPEDEEIKQKLIDSLLNLLGSYPPIPPPSFKLEISPAFLELVQLVIDLHPNDEEVRKELIENLISRIGKSDNVPPETISLDISEAALELFEWIFEKFPDDEDVHRRLIESFINKRKFSSSSPLDTPSLDISERFIELVKYILEKYPEDEEIKQKLIDSLLNLLGSYPPIPPPSFKLEISPAFLELVQLVIDLHPNDEEVRKELIENLISRIGKSDNVPPETISLDISEAALELFEWIFEKFPDDEDVHRRLIESFINKRKFSSSSPLDTPSLDISERFIELVKYILEKYPEDEEIKQKLIDSLLNLLGSYPPIPPPSFKLEISPAFLELVQLVIDLHPNDEEVRKELIENLISRIGKSDNVPPETISLDISEAALELFEWIFEKFPDDEDVHRRLIESFINKRKFSSSSPLDTPSLDISERFIELVKYILEKYPEDEEIKQKLIDSLLNLLGSYPPIPPPSFKLEISPAFLELVQLVIDLHPNDEEVRKELIENLISRIGKSDNVPPETISLDISEAALELFEWIFEKFPDDEDVHRRLIESFINKRKFSSSSPLDTPSLDISERFIELVKYILEKYPEDEEIKQKLIDSLLNLLGSYPPIPPPSFKLEISPAFLELVQLVIDLHPNDEEVRKELIENLISRIGKSDNVPPETISLDISEAALELFEWIFEKFPDDEDVHRRLIESFINKRKFSSSSPLDTPSLDISERFIELVKYILEKYPEDEEIKQKLIDSLLNLLGSY

SEQ ID NO: 67 - protein, 1008 aa, synthetic construct
  REGION 1..1008: note = synthetic peptide
SKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSPSKEKLGIQQDLFEGIIATLLSHKDPRVLYLMVTILKLTGSSP

SEQ ID NO: 68 - protein, 1060 aa, synthetic construct
  REGION 1..1060: note = synthetic peptide
SLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTGSLGWKVLLHLDLTWYPGADYTIDHDDMTRAARALAHGFERAAHSFAEAIGSTG

SEQ ID NO: 69 - protein, 1050 aa, synthetic construct
  REGION 1..1050: note = synthetic peptide
EGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSPEGAAIAENLATAYQGIGETLPSLQDLRVLHLSVIFSAEGSSP

SEQ ID NO: 70 - protein, 1260 aa, synthetic construct
  REGION 1..1260: note = synthetic peptide
GERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKILGERTDNPYYIGLLLKHLGEGLKKNKKLEKLKLDLPVFTTEPNPILEEGFKLLGEGLANIESPLDLEIKIL

SEQ ID NO: 71 - protein, 1680 aa, synthetic construct
  REGION 1..1680: note = synthetic peptide
PSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVSTPSLTLNDFGDLGKGLGEGLQGMENLEKLQLTITLKLTVST